SEQUENCE LISTING (4 sequences)

SEQ ID NO: 1 - DNA, 3822 bp, synthetic construct
  source 1..3822: mol_type = other DNA
atgtttgtctttctggtcctgctgcccctggtctcctctcagtgcgtgaacctgactactcgaacccagctgccacccgcctacaccaattccttcacccggggcgtgtactatcctgacaaggtgtttagaagctccgtgctgcactctacacaggatctgtttctgccattctttagcaacgtgacctggttccacgccatccacgtgagcggcaccaatggcacaaagcggttcgacaatcccgtgctgccttttaacgatggcgtgtacttcgcctctaccgagaagagcaacatcatcagaggctggatctttggcaccacactggactccaagacacagtctctgctgatcgtgaacaatgccaccaacgtggtcatcaaggtgtgcgagttccagttttgtaatgatcccttcctgggcgtgtactatcacaagaacaataagagctggatggagtccgagtttagagtgtattctagcgccaacaattgcacatttgagtacgtgtcccagcctttcctgatggacctggagggcaagcagggcaatttcaagaacctgagggagttcgtgtttaagaatatcgatggctacttcaagatctactctaagcacacccccatcaacctggtgcgcgacctgcctcagggcttcagcgccctggagccactggtggatctgcctatcggcatcaacatcacccggtttcagacactgctggccctgcacagaagctacctgacacccggcgactcctctagcggatggaccgcaggagcagcagcctactatgtgggctatctgcagcctaggaccttcctgctgaagtacaacgagaatggcaccatcacagacgccgtggattgcgccctggatcctctgagcgagacaaagtgtacactgaagtcctttaccgtggagaagggcatctatcagacatccaatttcagggtgcagccaaccgagtctatcgtgcgctttcctaatatcacaaacctgtgcccatttggcgaggtgttcaacgcaaccaggttcgcaagcgtgtacgcatggaataggaagcgcatctctaactgcgtggccgactatagcgtgctgtacaactccgcctctttcagcacctttaagtgctatggcgtgtcccccacaaagctgaatgacctgtgctttaccaacgtgtacgccgattctttcgtgatcaggggcgacgaggtgcgccagatcgcacctggacagacaggcaagatcgccgactacaattataagctgccagacgatttcaccggctgcgtgatcgcctggaacagcaacaatctggattccaaagtgggcggcaactacaattatctgtaccggctgtttagaaagagcaatctgaagcccttcgagagggacatctctacagagatctaccaggccggcagcaccccttgcaatggcgtggagggctttaactgttatttcccactgcagtcctacggcttccagcccacaaacggcgtgggctatcagccttaccgcgtggtggtgctgagctttgagctgctgcacgcaccagcaacagtgtgcggacccaagaagtccaccaatctggtgaagaacaagtgcgtgaacttcaacttcaacggcctgaccggaacaggcgtgctgaccgagtccaacaagaagttcctgccatttcagcagttcggcagggacatcgcagataccacagacgccgtgcgcgacccacagaccctggagatcctggatatcacaccctgctctttcggcggcgtgagcgtgatcacaccaggaaccaatacaagcaaccaggtggccgtgctgtatcaggacgtgaattgtaccgaggtgcctgtggccatccacgccgatcagctgaccccaacatggcgggtgtacagcaccggctccaacgtgttccagacaagagcaggatgcctgatcggagcagagcacgtgaacaattcctatgagtgcgacatcccaatcggcgccggcatctgtgcctcttaccagacccagacaaactctccaaggagagcacggagcgtggcatcccagtctatcatcgcctataccatgtccctgggcgccgagaattctgtggcctactctaacaatagcatcgccatcccaaccaacttcacaatctctgtgaccacagagatcctgcccgtgtccatgaccaagacatctgtggactgcacaatgtatatctgtggcgattctaccgagtgcagcaacctgctgctgcagtacggcagcttttgtacccagctgaatagagccctgacaggcatcgccgtggagcaggataagaacacacaggaggtgttcgcccaggtgaagcagatctacaagaccccccctatcaaggactttggcggcttcaatttttcccagatcctgcctgatccatccaagccttctaagcggagcccgatcgaggacctgctgttcaacaaggtgaccctggccgatgccggcttcatcaagcagtatggcgattgcctgggcgacatcgcagcacgggacctgatctgtgcccagaagtttaatggcctgaccgtgctgccacccctgctgacagatgagatgatcgcacagtacacaagcgccctgctggcaggaaccatcacatccggatggaccttcggcgcaggaccggccctgcagatcccctttccgatgcagatggcctataggttcaacggcatcggcgtgacccagaatgtgctgtacgagaaccagaagctgatcgccaatcagtttaactccgccatcggcaagatccaggacagcctgtcctctacaccgtccgccctgggcaagctgcaggatgtggtgaatcagaacgcccaggccctgaataccctggtgaagcagctgagctccaacttcggcgccatctctagcgtgctgaatgatatcctgagcaggctggacccgccggaggcagaggtgcagatcgaccggctgatcacaggcagactgcagtctctgcagacctatgtgacacagcagctgatcagggcagcagagatcagggcaagcgccaatctggcagcaaccaagatgtccgagtgcgtgctgggccagtctaagagagtggacttttgtggcaagggctatcacctgatgtccttccctcagtctgccccacacggcgtggtgtttctgcacgtgacctacgtgcccgcccaggagaagaacttcaccacagcccctgccatctgccacgatggcaaggcccactttccaagggagggcgtgttcgtgtccaacggcacccactggtttgtgacacagcgcaatttctacgagccccagatcatcaccacagacaataccttcgtgagcggcaactgtgacgtggtcatcggcatcgtgaacaataccgtgtatgatccactgcagcccgagctggacagctttaaggaggagctggataagtacttcaagaatcacacctcccctgacgtggatctgggcgacatcagcggcatcaatgcctccgtggtgaacatccagaaggagatcgaccgcctgaacgaggtggccaagaatctgaacgagagcctgatcgatctgcaggagctgggcaagtatgagcagtacatcaagtggccatggtacatctggctgggcttcatcgccggcctgatcgccatcgtgatggtgaccatcatgctgtgctgtatgacatcctgctgttcttgcctgaagggctgctgtagctgtggctcctgctgtaagtttgacgaagacgatagcgagcctgtcctgaagggagtgaagctgcattacacctga

SEQ ID NO: 2 - DNA, 3822 bp, synthetic construct
  source 1..3822: mol_type = other DNA
atgtttgtcttcctggtcctgctgcccctggtctcatcacagtgcgtgaatctgactacccgaactcagctgccccccgcctataccaattccttcacccggggcgtgtactatcccgacaaggtgtttagaagctccgtgctgcactctacacaggatctgtttctgcctttctttagcaacgtgacctggttccacgccatccacgtgagcggcaccaatggcacaaagcggttcgacaatccagtgctgccctttaacgatggcgtgtacttcgcctctaccgagaagagcaacatcatcagaggctggatctttggcaccacactggactccaagacacagtctctgctgatcgtgaacaatgccaccaacgtggtcatcaaggtgtgcgagttccagttttgtaatgatccattcctgggcgtgtactatcacaagaacaataagagctggatggagtccgagtttagagtgtattctagcgccaacaattgcacatttgagtacgtgtcccagcccttcctgatggacctggagggcaagcagggcaatttcaagaacctgagggagttcgtgtttaagaatatcgatggctacttcaagatctactctaagcacaccccaatcaacctggtgcgcgacctgccacagggcttcagcgccctggagccactggtggatctgcccatcggcatcaacatcacccggtttcagacactgctggccctgcacagaagctacctgacaccaggcgactcctctagcggatggaccgcaggagctgccgcctactatgtgggctatctgcagcccaggaccttcctgctgaagtacaacgagaatggcaccatcacagacgcagtggattgcgccctggaccccctgagcgagacaaagtgtacactgaagtcctttaccgtggagaagggcatctatcagacatccaatttcagggtgcagcctaccgagtctatcgtgcgctttcccaatatcacaaacctgtgcccttttggcgaggtgttcaacgcaaccaggttcgccagcgtgtacgcatggaataggaagcgcatctctaactgcgtggccgactatagcgtgctgtacaactccgcctctttcagcacctttaagtgctatggcgtgtcccccacaaagctgaatgacctgtgctttaccaacgtgtacgccgattctttcgtgatcaggggcgacgaggtgcgccagatcgcaccaggacagacaggcaagatcgcagactacaattataagctgcctgacgatttcaccggctgcgtgatcgcctggaacagcaacaatctggattccaaagtgggcggcaactacaattatctgtaccggctgtttagaaagagcaatctgaagccattcgagagggacatctctacagagatctaccaggcaggcagcaccccatgcaatggagtggagggctttaactgttatttccctctgcagtcctacggcttccagccaacaaacggcgtgggctatcagccctaccgcgtggtggtgctgagctttgagctgctgcacgcacctgcaacagtgtgcggaccaaagaagtccaccaatctggtgaagaacaagtgcgtgaacttcaacttcaacggcctgaccggcacaggcgtgctgaccgagtccaacaagaagttcctgccttttcagcagttcggcagggacatcgcagataccacagacgccgtgcgcgaccctcagaccctggagatcctggacatcacaccatgctctttcggcggcgtgagcgtgatcacaccaggcaccaatacaagcaaccaggtggccgtgctgtatcaggacgtgaattgtaccgaggtgcccgtggcaatccacgcagatcagctgacccctacatggcgggtgtacagcaccggctccaacgtgttccagacaagagccggatgcctgatcggagcagagcacgtgaacaattcctatgagtgcgacatccctatcggcgccggcatctgtgcctcttaccagacccagacaaactctccaaggagagcacggagcgtggcctcccagtctatcatcgcctataccatgtccctgggcgccgagaacagcgtggcctactctaacaatagcatcgccatccctaccaacttcacaatctctgtgaccacagagatcctgccagtgtccatgaccaagacatctgtggactgcacaatgtatatctgtggcgattctaccgagtgcagcaacctgctgctgcagtacggcagcttttgtacccagctgaatagagccctgacaggcatcgccgtggagcaggataagaacacacaggaggtgttcgcccaggtgaagcagatctacaagaccccccctatcaaggactttggcggcttcaatttttcccagatcctgcccgatccttccaagccctctaagcggagctttatcgaggacctgctgttcaacaaggtgaccctggccgatgccggcttcatcaagcagtatggcgattgcctgggcgacatcgcagcccgggacctgatctgtgcccagaagtttaatggcctgaccgtgctgccacccctgctgacagatgagatgatcgcacagtacacaagcgccctgctggccggcaccatcacatccggatggaccttcggcgcaggagccgccctgcagatcccctttgccatgcagatggcctataggttcaacggcatcggcgtgacccagaatgtgctgtacgagaaccagaagctgatcgccaatcagtttaactccgccatcggcaagatccaggacagcctgtcctctacagcctccgccctgggcaagctgcaggatgtggtgaatcagaacgcccaggccctgaataccctggtgaagcagctgagctccaacttcggcgccatctctagcgtgctgaatgacatcctgagccggctggaccccccagaggcagaggtgcagatcgaccggctgatcacaggcagactgcagtctctgcagacctatgtgacacagcagctgatcagggcagcagagatcagggccagcgccaatctggcagcaaccaagatgtccgagtgcgtgctgggccagtctaagagagtggacttttgtggcaagggctatcacctgatgtccttcccacagtctgcccctcacggcgtggtgtttctgcacgtgacctacgtgccagcccaggagaagaacttcaccacagcaccagcaatctgccacgatggcaaggcacactttcctagggagggcgtgttcgtgtccaacggcacccactggtttgtgacacagcgcaatttctacgagccacagatcatcaccacagacaataccttcgtgagcggcaactgtgacgtggtcatcggcatcgtgaacaataccgtgtatgatcctctgcagccagagctggacagctttaaggaggagctggataagtacttcaagaatcacacctcccccgacgtggatctgggcgacatcagcggcatcaatgcctccgtggtgaacatccagaaggagatcgaccgcctgaacgaggtggccaagaatctgaacgagagcctgatcgatctgcaggagctgggcaagtatgagcagtacatcaagtggccttggtacatctggctgggcttcatcgccggcctgatcgccatcgtgatggtgaccatcatgctgtgctgtatgacatcctgctgttcttgcctgaagggctgctgtagctgtggctcctgctgtaagtttgatgaggacgactctgaacctgtgctgaagggcgtgaagctgcattacacctaa

SEQ ID NO: 3 - DNA, 5439 bp, synthetic construct
  source 1..5439: mol_type = other DNA
atgtttgtctttctggtcctgctgcccctggtctcctcccagtgcgtgaacctgaccacccgcacccagctgccgcccgcctacaccaacagcttcacccgcggcgtgtactacccggacaaggtgttccgctccagcgtgctgcacagcacccaggacctgttcctgcccttcttctccaacgtgacctggttccacgccatccacgtgtccggcaccaacggcaccaagcgcttcgacaaccccgtgctgccgttcaacgacggcgtgtacttcgccagcaccgagaagtccaacatcatccgcggctggatcttcggcaccaccctggacagcaagacccagtccctgctgatcgtgaacaacgccaccaacgtggtgatcaaggtgtgcgagttccagttctgcaacgacccgttcctgggcgtgtactaccacaagaacaacaagagctggatggagtccgagttccgcgtgtactccagcgccaacaactgcaccttcgagtacgtgagccagcccttcctgatggacctggagggcaagcagggcaacttcaagaacctgcgcgagttcgtgttcaagaacatcgacggctacttcaagatctacagcaagcacaccccgatcaacctggtgcgcgacctgccccagggcttctcggccctggagcccctggtggacctgccgatcggcatcaacatcacccgcttccagaccctgctggccctgcaccgctcctacctgacccccggcgactcgtcctcgggctggaccgccggcgccgccgcctactacgtgggctacctgcagccccgcaccttcctgctgaagtacaacgagaacggcaccatcaccgacgccgtggactgcgccctggaccccctgtcggagacgaagtgcaccctgaagtccttcaccgtggagaagggcatctaccagaccagcaacttccgcgtgcagcccaccgagtccatcgtgcgcttccccaacatcaccaacctgtgcccgttcggcgaggtgttcaacgccacccgcttcgcctcggtgtacgcctggaaccgcaagcgcatcagcaactgcgtggccgactactccgtgctgtacaacagcgcctccttcagcacctttaagtgctacggcgtgagcccgaccaagctgaacgacctgtgcttcaccaacgtgtacgccgactcgttcgtgatccgcggcgacgaggtgcgccagatcgcccccggccagaccggcaagatcgccgactacaactacaagctgcccgacgacttcaccggctgcgtgatcgcctggaacagcaacaacctggactccaaggtgggcggcaactacaactacctgtaccgcctgttccgcaagagcaacctgaagccgttcgagcgcgacatcagcaccgagatctaccaggccggctccaccccctgcaacggcgtggagggcttcaactgctacttccccctgcagtcctacggcttccagcccaccaacggcgtgggctaccagccgtaccgcgtggtggtgctgagcttcgagctgctgcacgcgccggccaccgtgtgcggcccgaagaagtccaccaacctggtgaagaacaagtgcgtgaacttcaacttcaacggcctgaccggcaccggcgtgctgaccgagagcaacaagaagttcctgccgttccagcagttcggccgcgacatcgccgacaccaccgacgccgtgcgcgacccgcagaccctggagatcctggacatcaccccctgcagcttcggcggcgtgtccgtgatcaccccgggcaccaacacctccaaccaggtggccgtgctgtaccaggacgtgaactgcaccgaggtgcccgtggccatccacgccgaccagctgacccccacctggcgcgtgtactccaccggctcgaacgtgttccagacccgcgccggctgcctgatcggcgccgagcacgtgaacaacagctacgagtgcgacatccccatcggcgccggcatctgcgcctcctaccagacccagaccaacagcccccgccgcgcccgctcggtggcctcccagagcatcatcgcctacaccatgagcctgggcgccgagaactccgtggcctactccaacaacagcatcgccatcccgaccaacttcaccatcagcgtgaccaccgagatcctgcccgtgagcatgaccaagacctccgtggactgcaccatgtacatctgcggcgacagcaccgagtgctccaacctgctgctgcagtacggctccttctgcacccagctgaaccgcgccctgaccggcatcgccgtggagcaggacaagaacacccaagaggtgttcgcccaggtgaagcagatctacaagaccccgcccatcaaggacttcggtggcttcaacttctcccagatcctgccggacccctccaagcccagcaagcgctccccgatcgaggacctgctgttcaacaaggtgaccctggccgacgccggcttcatcaagcagtacggcgactgcctgggcgacatcgccgcccgcgacctgatctgcgcccagaagttcaacggcctgaccgtgctgcccccgctgctgaccgacgagatgatcgcccagtacacctcggccctgctggccggcaccatcacctcgggctggaccttcggcgccggccccgccctgcagatccccttcccgatgcagatggcctaccgcttcaacggcatcggcgtcacccagaacgtgctgtacgagaaccagaagctgatcgccaaccagttcaacagcgccatcggcaagatccaggactccctgagctccaccccctcggccctgggcaagctgcaggacgtggtgaaccagaacgcccaggccctgaacaccctggtgaagcagctgagctccaacttcggcgccatcagctccgtgctgaacgacatcctgtcgcgcctggacccgcccgaggccgaggtgcagatcgaccgcctgatcaccggccgcctgcagagcctgcagacctacgtgacccagcagctgatccgcgccgccgagatccgcgcctccgccaacctggccgccaccaagatgtcggagtgcgtgctgggccagtccaagcgcgtggacttctgcggcaagggctaccacctgatgtcgttcccccagtcggccccgcacggcgtggtgttcctgcacgtgacctacgtgccggcccaggagaagaacttcaccaccgccccggccatctgccacgacggcaaggcccacttcccccgcgagggcgtgttcgtgagcaacggcacccactggttcgtgacccagcgcaacttctacgagccccagatcatcaccaccgacaacaccttcgtgtccggcaactgcgacgtggtgatcggcatcgtgaacaacaccgtgtacgaccccctgcagcccgagctggacagcttcaaagaggagctggacaagtacttcaagaaccacacctcgcccgacgtggacctgggcgacatcagcggcatcaacgcctccgtggtgaacatccagaaggagatcgaccgcctgaacgaggtggccaagaacctgaacgagtccctgatcgacctgcaggagctgggcaagtacgagcagtacatcaagtggccctggtacatctggctgggcttcatcgccggcctgatcgccatcgtgatggtgaccatcatgctgtgctgcatgaccagctgctgctcctgcctgaagggctgctgcagctgcggctcctgctgcaagttcgacgaggacgactccgagcccgtgctgaagggggtcaagctgcactacaccctcgagggctccggcgagggcaggggaagtcttctaacatgcggggacgtggaggaaaatcccggcccaacgcgtgccaccatggcaaccgacattgacatgctgattgacctgggactggacctgagtgattccgacctggacgaagaccctcctgagcctgctgaaagccggagagacgatctggagtccgacagctccggcgagtgctctagctccgacgaggatatggaggaccctcacggagaggatggaccagagccaatcctggacgcagccagaccagccgtgcgcccctctcggcctgaggatccaggagtgccaagcacccagacacctagaccaaccgagaggcagggaccaaacgatccacagcctgcaccacactccgtgtggtctaggctgggagcaaggcgcccatcctgttctcccgagcagcacggaggcaaggtggcaaggctgcagccacctccaacaaaggcacagccagcaaggggaggccggagaggcaggcgccggggcagaggcaggggcggccctggcgccgccgacggcctgtccgatcctagaaggcgcgcaccaagaaccaacaggaatcctggaggaccccgccctggagcaggatggacagacggccctggcgccccacacggagaggcatggagaggaagcgagcagccagatccacctggaggacagaggaccaggggcgtgaggcaggccccaccccctctgatgacactggccatcgcaccaccacctgcagacccaagagcaccagcccccgagaggaaggcacctgcagcagacaccatcgatgcaaccacacgcctggtgctgcggagcatctccgagagagccgccgtggacaggatctctgagagctttggcagatctgcccaggtcatgcacgatccattcggcggccagccttttccagccgccaacagcccctgggcacctgtgctggcaggacagggaggacccttcgatgcagagacaaggagagtgtcctgggagacactggtggcacacggaccatctctgtacagaacctttgccggaaacccaagggcagcatccacagcaaaggcaatgagggactgcgtgctgaggcaggagaatttcatcgaggccctggcctctgccgatgagacactggcctggtgcaagatgtgcatccaccacaacctgccactgcggccccaggaccctatcatcggaaccgccgccgccgtgctggataatctggccacaagactgaggccttttctgcagtgttatctgaaggcaaggggcctgtgcggcctggacgagctgtgcagcaggcgccggctggcagacatcaaggatatcgcctccttcgtgtttgtgatcctggcaaggctggcaaatagggtggagaggggagtggcagagatcgactacgccacactgggagtgggagtgggcgagaagatgcacttctatctgccaggagcatgcatggcaggcctgatcgagatcctggatacccaccgccaggagtgctctagccgggtgtgcgagctgacagcctcccacattgtcgcccctccatacgtccacggaaaatacttctactgtaactccctgttctga

SEQ ID NO: 4 - DNA, 5160 bp, synthetic construct
  source 1..5160: mol_type = other DNA
atgtttgtctttctggtcctgctgcccctggtctcctctcagtgcgtgaacctgactactcgaacccagctgccacccgcctacaccaattccttcacccggggcgtgtactatcctgacaaggtgtttagaagctccgtgctgcactctacacaggatctgtttctgccattctttagcaacgtgacctggttccacgccatccacgtgagcggcaccaatggcacaaagcggttcgacaatcccgtgctgccttttaacgatggcgtgtacttcgcctctaccgagaagagcaacatcatcagaggctggatctttggcaccacactggactccaagacacagtctctgctgatcgtgaacaatgccaccaacgtggtcatcaaggtgtgcgagttccagttttgtaatgatcccttcctgggcgtgtactatcacaagaacaataagagctggatggagtccgagtttagagtgtattctagcgccaacaattgcacatttgagtacgtgtcccagcctttcctgatggacctggagggcaagcagggcaatttcaagaacctgagggagttcgtgtttaagaatatcgatggctacttcaagatctactctaagcacacccccatcaacctggtgcgcgacctgcctcagggcttcagcgccctggagccactggtggatctgcctatcggcatcaacatcacccggtttcagacactgctggccctgcacagaagctacctgacacccggcgactcctctagcggatggaccgcaggagcagcagcctactatgtgggctatctgcagcctaggaccttcctgctgaagtacaacgagaatggcaccatcacagacgccgtggattgcgccctggatcctctgagcgagacaaagtgtacactgaagtcctttaccgtggagaagggcatctatcagacatccaatttcagggtgcagccaaccgagtctatcgtgcgctttcctaatatcacaaacctgtgcccatttggcgaggtgttcaacgcaaccaggttcgcaagcgtgtacgcatggaataggaagcgcatctctaactgcgtggccgactatagcgtgctgtacaactccgcctctttcagcacctttaagtgctatggcgtgtcccccacaaagctgaatgacctgtgctttaccaacgtgtacgccgattctttcgtgatcaggggcgacgaggtgcgccagatcgcacctggacagacaggcaagatcgccgactacaattataagctgccagacgatttcaccggctgcgtgatcgcctggaacagcaacaatctggattccaaagtgggcggcaactacaattatctgtaccggctgtttagaaagagcaatctgaagcccttcgagagggacatctctacagagatctaccaggccggcagcaccccttgcaatggcgtggagggctttaactgttatttcccactgcagtcctacggcttccagcccacaaacggcgtgggctatcagccttaccgcgtggtggtgctgagctttgagctgctgcacgcaccagcaacagtgtgcggacccaagaagtccaccaatctggtgaagaacaagtgcgtgaacttcaacttcaacggcctgaccggaacaggcgtgctgaccgagtccaacaagaagttcctgccatttcagcagttcggcagggacatcgcagataccacagacgccgtgcgcgacccacagaccctggagatcctggatatcacaccctgctctttcggcggcgtgagcgtgatcacaccaggaaccaatacaagcaaccaggtggccgtgctgtatcaggacgtgaattgtaccgaggtgcctgtggccatccacgccgatcagctgaccccaacatggcgggtgtacagcaccggctccaacgtgttccagacaagagcaggatgcctgatcggagcagagcacgtgaacaattcctatgagtgcgacatcccaatcggcgccggcatctgtgcctcttaccagacccagacaaactctccaaggagagcacggagcgtggcatcccagtctatcatcgcctataccatgtccctgggcgccgagaattctgtggcctactctaacaatagcatcgccatcccaaccaacttcacaatctctgtgaccacagagatcctgcccgtgtccatgaccaagacatctgtggactgcacaatgtatatctgtggcgattctaccgagtgcagcaacctgctgctgcagtacggcagcttttgtacccagctgaatagagccctgacaggcatcgccgtggagcaggataagaacacacaggaggtgttcgcccaggtgaagcagatctacaagaccccccctatcaaggactttggcggcttcaatttttcccagatcctgcctgatccatccaagccttctaagcggagcccgatcgaggacctgctgttcaacaaggtgaccctggccgatgccggcttcatcaagcagtatggcgattgcctgggcgacatcgcagcacgggacctgatctgtgcccagaagtttaatggcctgaccgtgctgccacccctgctgacagatgagatgatcgcacagtacacaagcgccctgctggcaggaaccatcacatccggatggaccttcggcgcaggaccggccctgcagatcccctttccgatgcagatggcctataggttcaacggcatcggcgtgacccagaatgtgctgtacgagaaccagaagctgatcgccaatcagtttaactccgccatcggcaagatccaggacagcctgtcctctacaccgtccgccctgggcaagctgcaggatgtggtgaatcagaacgcccaggccctgaataccctggtgaagcagctgagctccaacttcggcgccatctctagcgtgctgaatgatatcctgagcaggctggacccgccggaggcagaggtgcagatcgaccggctgatcacaggcagactgcagtctctgcagacctatgtgacacagcagctgatcagggcagcagagatcagggcaagcgccaatctggcagcaaccaagatgtccgagtgcgtgctgggccagtctaagagagtggacttttgtggcaagggctatcacctgatgtccttccctcagtctgccccacacggcgtggtgtttctgcacgtgacctacgtgcccgcccaggagaagaacttcaccacagcccctgccatctgccacgatggcaaggcccactttccaagggagggcgtgttcgtgtccaacggcacccactggtttgtgacacagcgcaatttctacgagccccagatcatcaccacagacaataccttcgtgagcggcaactgtgacgtggtcatcggcatcgtgaacaataccgtgtatgatccactgcagcccgagctggacagctttaaggaggagctggataagtacttcaagaatcacacctcccctgacgtggatctgggcgacatcagcggcatcaatgcctccgtggtgaacatccagaaggagatcgaccgcctgaacgaggtggccaagaatctgaacgagagcctgatcgatctgcaggagctgggcaagtatgagcagtacatcaagtggccatggtacatctggctgggcttcatcgccggcctgatcgccatcgtgatggtgaccatcatgctgtgctgtatgacatcctgctgttcttgcctgaagggctgctgtagctgtggctcctgctgtaagtttgacgaagacgatagcgagcctgtcctgaagggagtgaagctgcattacaccctcgagggctccggcgagggcaggggaagtcttctaacatgcggggacgtggaggaaaatcccggcccaacgcgtgccaccatgagcgacaatggaccccagaatcagagaaacgcaccccgaatcacttttggcggacccagcgacagcacaggcagtaatcagaacggagagcggagcggagcaagatccaagcagagacggccccagggcctgccaaacaataccgcatcctggttcaccgccctgacacagcacggcaaggaggacctgaagtttccaaggggacagggagtgcctatcaacaccaatagctcccctgacgatcagatcggctactataggagggcaacaaggagaatcaggggaggcgacggcaagatgaaggatctgagcccacgctggtacttctactatctgggaaccggacctgaggcaggcctgccatatggcgccaacaaggacggaatcatctgggtggcaaccgagggcgccctgaacacaccaaaggatcacatcggcacaagaaatcccgccaacaatgcagcaatcgtgctgcagctgccacagggaaccacactgcccaagggcttttacgcagagggctctcggggaggcagccaggcatctagcagatcctctagccggagcagaaactcctctaggaattccaccccaggaagctccaggggcacatcccctgcccgcatggcaggaaacggaggcgacgccgccctggccctgctgctgctggatcgcctgaatcagctggagtccaagatgtctggcaagggacagcagcagcagggacagaccgtgacaaagaagtccgccgccgaggcctctaagaagccaaggcagaagcgcaccgccacaaaggcctacaacgtgacccaggccttcggcaggcgcggaccagagcagacacagggcaattttggcgaccaggagctgatcaggcagggaaccgattataagcactggcctcagatcgcccagttcgccccatctgccagcgccttctttggcatgtctagaatcggcatggaggtgacccccagcggcacatggctgacctacacaggcgccatcaagctggacgataaggaccctaacttcaaggatcaggtcatcctgctgaacaagcacatcgacgcctataagacctttccccctacagagcccaagaaggacaagaagaagaaggccgatgagacacaggccctgcctcagaggcagaagaagcagcagaccgtgacactgctgccagccgccgatctggacgatttctccaaacagctgcagcagtcaatgtcatccgccgattccactcaggcataa

(19) United States
(12) Patent Application Publication
(10) Pub. No.: US 2025/0000966 A1
(43) Pub. Date: Jan. 2, 2025

(54) MRNA VACCINE DESIGN VIA THE ALTERATION OF CODON USAGE

(71) Applicant: The Cleveland Clinic Foundation, Cleveland, OH (US)

(72) Inventors: Jae U. Jung, Cleveland, OH (US); Chih-Jen Lai, Cleveland, OH (US)

(21) Appl. No.: 18/701,156

(22) PCT Filed: Oct. 13, 2022
(86) PCT No.: PCT/US22/78052

§ 371 Date: Apr. 12, 2024

Related U.S. Application Data
(60) Provisional application No. 63/255,072, filed on Oct. 13, 2021.

(51) Int. Cl. A61K 39/215; A61K 39/00; A61P 37/04; C07K 14/005

(52) U.S. Cl. CPC A61K 39/215; A61P 37/04; C07K 14/005; A61K 2039/53; A61K 2039/575; C12N 2710/16022; C12N 2770/20022; C12N 2770/20034; C12N 2770/20071

(57) ABSTRACT

The present disclosure provides compositions and polynucleotides for increasing expression of an immunogenic and/or antigenic polypeptide (e.g., in a vaccine). The disclosure further provides methods of using the disclosed compositions, polynucleotides, and vaccines for the treatment of diseases and disorders (e.g., infections). The compositions and polynucleotides include a first nucleic acid encoding a viral regulatory protein and a second nucleic acid encoding an immunogenic polypeptide, wherein the immunogenic polypeptide is codon-optimized to a vims from which the regulatory protein is derived.

### CROSS-REFERENCE TO RELATED APPLICATIONS

This application claims the benefit of U.S. Provisional Application No. 63/255,072, filed Oct. 13, 2021, the contents of which are herein incorporated by reference in their entirety.

### STATEMENT REGARDING FEDERAL FUNDING

This invention was made with government support under AI152190 and AI140705 awarded by the National Institutes of Health. The government has certain rights in the invention.

### FIELD

The present disclosure provides compositions, sequences, and formulations for vaccines (e.g., mRNA based vaccines) which result in increased expression of an immunogenic and/or antigenic polypeptide.

### SEQUENCE LISTING STATEMENT

The contents of the electronic sequence listing titled CCF-39983-601.xml (Size: 22,724 bytes; and Date of Creation: Oct. 13, 2022) is herein incorporated by reference in its entirety.

### BACKGROUND

Recent advances in prophylactics and therapeutics have focused on the introduction of foreign nucleic acids, deoxyribonucleic acid (DNA) and ribonucleic acid (RNA) molecules, encoding one or more polypeptides to provide a therapeutic benefit. For example, nucleic acid therapeutics have emerged as promising alternatives to conventional vaccine approaches. mRNA vaccines represent a promising alternative to conventional vaccine approaches because of their high potency, capacity for rapid development and potential for low-cost manufacture and safe administration.

### SUMMARY

Disclosed herein are compositions and polynucleotides comprising a first nucleic acid encoding a viral regulatory protein and a second nucleic acid encoding an immunogenic polypeptide, wherein the immunogenic polypeptide is codon-optimized to a virus from which the regulatory protein is derived.

In some embodiments, the regulatory protein comprises an immediate-early viral protein. In some embodiments, the regulatory protein comprises an ICP27 protein. In some embodiments, the ICP27 protein is derived from herpes simplex virus (HSV). In some embodiments, the regulatory protein comprises an ORF57 protein. In some embodiments, the ORF57 protein is derived from Kaposi's sarcoma-associated herpesvirus (KSHV) or rhesus monkey rhadinovirus (RRV).

In some embodiments, the immunogenic polypeptide is derived from a pathogen or infectious agent. In some embodiments, the immunogenic polypeptide comprises a viral structural protein. In some embodiments, the immunogenic polypeptide comprises a viral spike protein or a fragment or variant thereof. In some embodiments, the viral spike protein or a fragment or variant thereof is from a coronavirus. In some embodiments, the coronavirus is SARS-CoV-2.

In some embodiments, the regulatory protein and the immunogenic polypeptide are derived from different viruses.

In some embodiments, the regulatory protein comprises HSV ICP27 protein and the viral immunogenic polypeptide comprises SARS-CoV-2 spike protein or a fragment or variant thereof codon-optimized to HSV glycoprotein B.

In some embodiments, the regulatory protein comprises RRV ORF57 protein and the viral immunogenic polypeptide comprises SARS-CoV-2 spike protein or a fragment or variant thereof codon-optimized to RRV glycoprotein B.

In some embodiments, a single polynucleotide comprises the first nucleic acid and the second nucleic acid. In some embodiments, the single polynucleotide is mRNA. In some embodiments, the single polynucleotide encodes the mRNA.

In some embodiments, the polynucleotide further comprises a ribosome skipping peptide sequence or a sequence encoding thereof. In some embodiments, the ribosome skipping peptide comprises a 2A family peptide.

In some embodiments, the polynucleotide further comprises or encodes: a 5′ untranslated region (UTR), a 5′ cap, a 3′ UTR, a 3′ tailing sequence or any combination thereof. In some embodiments, the 3′ tailing sequence comprises a polyA tail, a polyG quartet, a stem loop sequence, a triple helix forming sequence, a tRNA-like sequence, or any combination thereof.

Also disclosed herein are vaccines comprising the composition or the polynucleotides provided herein and at least one adjuvant, a delivery vehicle, or a combination thereof.

In some embodiments, the delivery vehicle comprises a lipid nanoparticle encapsulating the composition or polynucleotide. In some embodiments, the lipid nanoparticle comprises a cationic lipid, a neutral and/or non-cationic lipid, a sterol, or any combination thereof. In some embodiments, the non-cationic lipid comprises a phospholipid. In some embodiments, the sterol comprises cholesterol or a modification or ester thereof. In some embodiments, the lipid nanoparticle comprises a polyethylene glycol (PEG)-lipid conjugate.

Further disclosed herein are methods of treating or preventing a disease or disorder and method of inducing an immune response comprising administering the compositions, polynucleotides, or vaccines provided herein to a subject in need thereof. In some embodiments, the disease or disorder comprises an infection. In some embodiments, the infection comprises a coronavirus infection. In some embodiments, the coronavirus is severe acute respiratory syndrome coronavirus-2 (SARS-CoV-2).

In some embodiments, the administering comprises an initial immunization and at least one subsequent immunization.

In some embodiments, the subject is human.

Other aspects and embodiments of the disclosure will be apparent in light of the following detailed description and accompanying figures.

### BRIEF DESCRIPTION OF THE DRAWINGS

FIGS. 1A and 1B are comparisons of codon usage in SARS-CoV-2 S. RRV glycoprotein B (gB), and HSV-1 gB. To generate codon-modified (gB-like) SARS-CoV-2 S, some codons of SARS-CoV-2 S (empty colored circles) were changed into corresponding synonymous codons (filled colored circles) to reflect the codon usage of gB.

FIG. 2 are schematics of exemplary virus codon optimization and regulatory factor mRNA constructs.

FIGS. 3A and 3B are codon optimized spike protein expression in multiple viral systems. FIG. 3A shows spike codon optimization to RRVgB or HSVgB which increase S level. S-2P—N vector significantly elevates S expression as well. FIG. 3B are graphs showing the quantitation of protein levels in FIG. 3A. Asterisks *, **, *** represent statistically significant different of p value <0.05, p values <0.01, and p value <0.001, respectively.

FIGS. 4A and 4B are codon optimized spike protein expression in an HSV codon optimization system compared to S2P. FIG. 4A shows spike protein variants (S2P; full-length Spike protein with 2 proline mutation or S6P; full-length Spike protein with 6 proline mutation) changes when co-expressed with ICP27. FIG. 4B are graphs showing the quantitation of protein levels in FIG. 4A. Asterisks *, **, *** represent statistically significant different of p value <0.05, p values <0.01, and p value <0.001, respectively.

FIGS. 5A and 5B are codon optimized spike protein expression in an RRV codon optimization system compared to S2P. FIG. 5A shows spike protein variants (S2P; full-length Spike protein with 2 proline mutation or S6P; full-length Spike protein with 6 proline mutation) changes when co-expressed with ORF57. FIG. 5B are graphs showing the quantitation of protein levels in FIG. 5A. Asterisks *, **, *** represent statistically significant different of p value <0.05, p values <0.01, and p value <0.001, respectively.

FIG. 6 is a schematic of an exemplary experimental plan to determine the humoral response and cellular response induced by mRNA vaccines as described herein.

FIGS. 7A and 7B are expression for spike protein variants. FIG. 7A shows the expression level changes of spike protein variants (S2P: full-length Spike protein with 2 proline mutation or S6P; full-length Spike protein with 6 proline mutation) when co-expressed with ORF57 or ICP27.

FIG. 7B are graphs showing the quantitation of protein levels in FIG. 7A. Asterisks ** and *** represent statistically significant different p value <0.01, and p value <0.001, respectively.

FIGS. 8A-8B show spike surface expression with mRNA transfection as determined by fluorescence-activated cell sorting (FACS) (FIGS. 8A and 8B) and western blot (FIG. 8C).

FIGS. 9A and 9B show spike protein stability following mRNA transfection, as determined by cycloheximide chase assay. FIG. 9A is a western blot of expression levels following transfection with S-6P variant co expressed with ICP27 or SARS-CoV-2 N. FIG. 9B is a graph showing the quantitation of the protein levels in FIG. 9A.

FIG. 10 is graphs of the level of IgG (left) or neutralizing (right) antibodies from mice serum against wild-type Spike, as determined by endpoint ELISA or pseudovirus neutralization assay.

FIGS. 11A and 11B is graphs of the level of IgG (FIG. 11A) or neutralizing (FIG. 11B) antibodies from mice against wild-type Spike, as determined by endpoint ELISA or pseudovirus neutralization assay.

### DETAILED DESCRIPTION

The present disclosure provides compositions, sequences, and formulations for designing and optimizing the expression of an immunogenic polypeptide (e.g., an antigen) in mRNA vaccines (e.g., SARS-CoV-2 spike protein and variants thereof) by altering preferred codon usages and co-expressing with an immediate-early regulatory protein (e.g., ICP 27 from HSV, ORF57 from RRV) using the same skewed codon usage. The compositions and formulations comprise a polynucleotide (e.g., mRNA) encoding a regulatory protein and an immunogenic polypeptide using the skewed viral codon of the regulatory protein virus.

The compositions and polynucleotides increased expression of an immunogenic and/or antigenic polypeptide. Expression of ICP27 or ORF57 proteins highly elevated SARS-CoV-2 spike protein expression when the spike protein codons were optimized to the skewed codon usage of the regulatory proteins (e.g., HSV glycoprotein B or RRV glycoprotein B usages, respectively). Due to the increased expression levels of the immunogenic polypeptide in these mRNA vaccine constructs, lower dosages or decreased need for booster immunizations may be facilitated.

Section headings as used in this section and the entire disclosure herein are merely for organizational purposes and are not intended to be limiting.

### 1. Definitions

The terms “comprise(s).” “include(s),” “having,” “has,” “can,” “contain(s),” and variants thereof, as used herein, are intended to be open-ended transitional phrases, terms, or words that do not preclude the possibility of additional acts or structures. As used herein, comprising a certain sequence or a certain SEQ ID NO usually implies that at least one copy of said sequence is present in recited peptide or polynucleotide. However, two or more copies are also contemplated. The singular forms “a,” “and” and “the” include plural references unless the context clearly dictates otherwise. The present disclosure also contemplates other embodiments “comprising,” “consisting of” and “consisting essentially of,” the embodiments or elements presented herein, whether explicitly set forth or not.

For the recitation of numeric ranges herein, each intervening number there between with the same degree of precision is explicitly contemplated. For example, for the range of 6-9, the numbers 7 and 8 are contemplated in addition to 6 and 9, and for the range 6.0-7.0, the number 6.0, 6.1, 6.2, 6.3, 6.4, 6.5, 6.6, 6.7, 6.8, 6.9, and 7.0 are explicitly contemplated.

Unless otherwise defined herein, scientific, and technical terms used in connection with the present disclosure shall have the meanings that are commonly understood by those of ordinary skill in the art. The meaning and scope of the terms should be clear; in the event, however of any latent ambiguity, definitions provided herein take precedent over any dictionary or extrinsic definition. Further, unless otherwise required by context, singular terms shall include pluralities and plural terms shall include the singular.

The term “immunization,” as used herein, refers to a process that increases an organisms' reaction to antigen and therefore improves its ability to resist or overcome infection.

“Polynucleotide” or “oligonucleotide” or “nucleic acid,” as used herein, means at least two nucleotides covalently linked together. The polynucleotide may be DNA, both genomic and cDNA, RNA, or a hybrid, where the polynucleotide may contain combinations of deoxyribo- and ribo-nucleotides, and combinations of bases including uracil, adenine, thymine, cytosine, guanine, inosine, xanthine hypoxanthine, isocytosine and isoguanine. The nucleic acid, whether DNA or RNA may comprise non-natural nucleotides, modified nucleotides, and/or non-nucleotide building blocks that can exhibit the same function as natural nucleotides (e.g., “nucleotide analogs”). Nucleic acids may be obtained by chemical synthesis methods or by recombinant methods. Polynucleotides may be single- or double-stranded or may contain portions of both double stranded and single stranded sequence. The depiction of a single strand also defines the sequence of the complementary strand. Thus, a nucleic acid also encompasses the complementary strand of a depicted single strand. Many variants of a nucleic acid may be used for the same purpose as a given nucleic acid. Thus, a nucleic acid also encompasses substantially identical nucleic acids and complements thereof.

Nucleic acid or amino acid sequence “identity,” as described herein, can be determined by comparing a nucleic acid or amino acid sequence of interest to a reference nucleic acid or amino acid sequence. The percent identity is the number of nucleotides or amino acid residues that are the same (e.g., that are identical) as between the sequence of interest and the reference sequence divided by the length of the longest sequence (e.g., the length of either the sequence of interest or the reference sequence, whichever is longer). A number of mathematical algorithms for obtaining the optimal alignment and calculating identity between two or more sequences are known and incorporated into a number of available software programs. Examples of such programs include CLUSTAL-W, T-Coffee, and ALIGN (for alignment of nucleic acid and amino acid sequences), BLAST programs (e.g., BLAST 2.1, BL2SEQ, and later versions thereof) and FASTA programs (e.g., FASTA3x, FAS™, and SSEARCH) (for sequence alignment and sequence similarity searches). Sequence alignment algorithms also are disclosed in, for example, Altschul et al., J. Molecular Biol., 215(3): 403-410 (1990), Beigert et al., Proc. Natl. Acad. Sci. USA, 106(10): 3770-3775 (2009), Durbin et al., eds., Biological Sequence Analysis: Probabilistic Models of Proteins and Nucleic Acids, Cambridge University Press, Cambridge, UK (2009), Soding, Bioinformatics, 21(7): 951-960 (2005), Altschul et al., Nucleic Acids Res., 25(17): 3389-3402 (1997), and Gusfield, Algorithms on Strings, Trees and Sequences, Cambridge University Press, Cambridge UK (1997)).

A “peptide” or “polypeptide” is a linked sequence of two or more amino acids linked by peptide bonds. Peptides and polypeptides include proteins such as binding proteins, receptors, and antibodies. The terms “polypeptide” and “protein,” are used interchangeably herein.

The term “vaccine,” as used herein, refers to any pharmaceutical composition containing at least one antigenic or immunogenic peptide or other immunogen or at least one nucleic acid encoding at least one antigenic or immunogenic peptide or other immunogen, which can be used to prevent or treat a disease or condition in a subject.

As used herein, the term “preventing” refers to partially or completely delaying onset of an infection, disease, disorder and/or condition; partially or completely delaying onset of one or more symptoms, features, or manifestations of a particular infection, disease, disorder, and/or condition; partially or completely delaying progression from an infection, a particular disease, disorder and/or condition; and/or decreasing the risk of developing pathology associated with the infection, the disease, disorder, and/or condition.

As used herein, “treat,” “treating,” and the like means a slowing, stopping, or reversing of progression of a disease or disorder when provided a composition or vaccine described herein to an appropriate control subject. The term also means a reversing of the progression of such a disease or disorder to a point of eliminating or greatly reducing the cell proliferation. As such. “treating” means an application or administration of the compositions described herein to a subject, where the subject has a disease or a symptom of a disease, where the purpose is to cure, heal, alleviate, relieve, alter, remedy, ameliorate, improve, or affect the disease or symptoms of the disease.

A “subject” or “patient” may be human or non-human and may include, for example, animal strains or species used as “model systems” for research purposes, such a mouse model as described herein. Likewise, patient may include either adults or juveniles (e.g., children). Moreover, patient may mean any living organism, preferably a mammal (e.g., a human or a non-human) that may benefit from the administration of compositions contemplated herein. Examples of mammals include, but are not limited to, any member of the Mammalian class: humans, non-human primates such as chimpanzees, and other apes and monkey species; farm animals such as cattle, horses, sheep, goats, swine; domestic animals such as rabbits, dogs, and cats: laboratory animals including rodents, such as rats, mice and guinea pigs, and the like. Examples of non-mammals include, but are not limited to, birds, fish, and the like. In one embodiment of the methods and compositions provided herein, the mammal is a human.

As used herein, the terms “providing,” “*administering.” and “introducing,” are used interchangeably herein and refer to the placement of the compositions, polynucleotides, or vaccines of the disclosure into a subject by a method or route which results in at least partial localization to a desired site. Administration may be by any appropriate route which results in delivery to a desired location in the subject.

Preferred methods and materials are described below, although methods and materials similar or equivalent to those described herein can be used in practice or testing of the present disclosure. All publications, patent applications, patents and other references mentioned herein are incorporated by reference in their entirety. The materials, methods, and examples disclosed herein are illustrative only and not intended to be limiting.

### 2. Compositions and Polynucleotides

Disclosed herein are compositions comprising a first nucleic acid encoding a regulatory protein and a second nucleic acid encoding an immunogenic and/or antigenic polypeptide. The compositions facilitate increased expression of the immunogenic and/or antigenic polypeptide.

The regulatory protein may be a viral regulatory protein or a fragment thereof. In some embodiments, the regulatory protein comprises an immediate-early viral protein or a fragment thereof. Viral-encoded regulatory proteins (e.g., Rev of HIV/SIV and ORF57 of herpesviruses) recognize the specific nature of the skewed codon usage to allow expression of their structural gene products. Skewed codon usage temporally regulates late expression of structural gene products and the transinducibility of expression of glycoproteins can be altered by changing the nature of the codon usage. Table 1 shows exemplary viral structural and regulatory proteins suitable for skewed codon usage. See for example, Shin et al. Proc Natl Acad Sci. 2015, incorporated herein by reference in its entirety.

TABLE 1

| | HIV/SIV | HSV-1 | RRV | KSHV | SARS-CoV-2 |
|---|---|---|---|---|---|
| Structural | Gp160 | gB/gH | gB/gH | gB/gH | Spike |
| Protein | | | | | |
| Regulatory | REV | ICP27 | ORF57 | ORF57 | N |
| Protein | | | | | |

In some embodiments, the regulatory protein comprises a HIV Rev protein, herpes simplex virus type 1 (HSV-1) ICP27 protein, rhesus monkey rhadinovirus (RRV) or Kaposi's sarcoma-associated herpesvirus (KSHV) ORF57 protein (also known as mRNA transcript accumulation (Mta)), or SARS-CoV-2 N protein.

The immunogenic and/or antigenic polypeptide may be any protein or polypeptide the body is able to use to boost immunity against pathogens and/or fight diseases such as cancer and rare genetic conditions. Thus, immunogenic and/or antigenic polypeptides include any polypeptides capable of stimulating or triggering an immune response. In some embodiments, the immunogenic and/or antigenic polypeptide is a polypeptide derived from a pathogen or infectious agent, e.g., a bacterium, virus, or other microorganism that can cause disease.

In some embodiments, the immunogenic and/or antigenic polypeptide is derived from a virus. In some embodiments, the immunogenic polypeptide comprises a viral structural protein, for example, a late structure protein. Viral structural proteins include: viral envelope proteins; viral membrane proteins; viral nucleocapsid proteins; and viral spike proteins.

In some embodiments, the immunogenic and/or antigenic polypeptide comprises a viral spike protein, or a fragment or variant thereof. The viral spike protein may be derived from any virus. The spike protein may be derived fully or partially from a respiratory virus. Respiratory viruses include, but are not limited to: influenza virus, respiratory syncytial virus, parainfluenza viruses, metapneumovirus, rhinovirus, coronaviruses, adenoviruses, and bocaviruses.

In some embodiments, the spike protein is derived from a coronavirus. The coronavirus family comprises 45 species distributed between four genera: alphacoronavirus, betacoronavirus, deltacoronavirus, and gammacoronavirus. In some embodiments, the spike protein is derived from a betacoronavirus.

In select embodiments, the spike protein is derived from SARS-CoV or SARS-CoV-2. In select embodiments, the spike protein is wild-type spike protein from SARS-CoV-2 (Accession #QHD43416), or a fragment thereof. However, the invention is not limited to this exemplary sequence. SARS-CoV-2 spike protein may comprise the wild-type amino acid sequence or variant having an amino acid sequence that is at least about 70% identical (e.g., about 70%, about 71%, about 72%, about 73%, about 74%, about 75%, about 76%, about 77%, about 78%, about 79%, about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, or about 100%) to the amino acid sequence of the wild-type spike protein.

The spike protein may comprise one or more (e.g., 1, 2, 3, 4, 5, 6, 7, 8, 9, 10, 15, 20, 25, 50, 100, 150, 200, etc.) amino acid substitutions. An amino acid “replacement” or “substitution” refers to the replacement of one amino acid at a given position or residue by another amino acid at the same position or residue within a polypeptide sequence. Amino acids are broadly grouped as “aromatic” or “aliphatic.” An aromatic amino acid includes an aromatic ring. Examples of “aromatic” amino acids include histidine (H or His), phenylalanine (F or Phe), tyrosine (Y or Tyr), and tryptophan (W or Trp). Non-aromatic amino acids are broadly grouped as “aliphatic.” Examples of “aliphatic” amino acids include glycine (G or Gly), alanine (A or Ala), valine (V or Val), leucine (L or Leu), isoleucine (I or Ile), methionine (M or Met), serine (S or Ser), threonine (T or Thr), cysteine (C or Cys), proline (P or Pro), glutamic acid (E or Glu), aspartic acid (A or Asp), asparagine (N or Asn), glutamine (Q or Gin), lysine (K or Lys), and arginine (R or Arg).

The amino acid replacement or substitution can be conservative, semi-conservative, or non-conservative. The phrase “conservative amino acid substitution” or “conservative mutation” refers to the replacement of one amino acid by another amino acid with a common property. A functional way to define common properties between individual amino acids is to analyze the normalized frequencies of amino acid changes between corresponding proteins of homologous organisms (Schulz and Schirmer, Principles of Protein Structure, Springer-Verlag, New York (1979)). According to such analyses, groups of amino acids may be defined where amino acids within a group exchange preferentially with each other, and therefore resemble each other most in their impact on the overall protein structure (Schulz and Schirmer, supra). Examples of conservative amino acid substitutions include substitutions of amino acids within the sub-groups described above, for example, lysine for arginine and vice versa such that a positive charge may be maintained, glutamic acid for aspartic acid and vice versa such that a negative charge may be maintained, serine for threonine such that a free —OH can be maintained, and glutamine for asparagine such that a free —NH₂ can be maintained. “Semi-conservative mutations” include amino acid substitutions of amino acids within the same groups listed above, but not within the same sub-group. For example, the substitution of aspartic acid for asparagine, or asparagine for lysine, involves amino acids within the same group, but different sub-groups. “Non-conservative mutations” involve amino acid substitutions between different groups, for example, lysine for tryptophan, or phenylalanine for serine, etc.

In some embodiments, the spike protein is a variant of a wild-type spike protein or fragment thereof. For example, the spike protein may comprise full-length SARS-CoV-2 spike protein with two proline mutations or six proline mutations. In some embodiments, the spike protein comprises the 2P variant of the SARS-CoV-2 spike protein (S2P) that contains two stabilizing proline substitutions at residues 986-987. In some embodiments, the S2P protein is encoded by a nucleic acid sequence that is at least about 70% identical to SEQ ID NO: 2, or an RNA equivalent thereof.

In some embodiments, the spike protein comprises the 6P variant of the SARS-CoV-2 spike protein (S6P), which has a more stabilized prefusion structure because of the introduction of four additional proline substitutions in the S2 segment of the spike protein. In some embodiments, the S6P protein is encoded by a nucleic acid sequence that is at least about 70% identical to SEQ ID NO: 1, or an RNA equivalent thereof.

In the compositions described herein, the nucleic acid encoding the immunogenic polypeptide is codon-optimized to the virus from which the regulatory protein is derived. In some embodiments, the regulatory protein and the immunogenic polypeptide are derived from the same or different viruses.

In select embodiments, the regulatory protein comprises RRV ORF57 and the viral immunogenic polypeptide comprises SARS-CoV-2 spike protein or a fragment or variant thereof codon-optimized to RRV glycoprotein B. In alternative embodiments, the regulatory protein comprises HSV ICP27 and the viral immunogenic polypeptide comprises SARS-CoV-2 spike protein or a fragment or variant thereof codon-optimized to HSV glycoprotein B. In some embodiments, the SARS-CoV-2 spike protein or a fragment or variant thereof comprises the S6P protein or the S2P protein codon-optimized to RRV glycoprotein B or HSV glycoprotein B.

In some embodiments, the first nucleic acid and second nucleic acid are on a single polynucleotide. The first nucleic acid and second nucleic acid may be provided on the single polynucleotide in any orientation (e.g., the first nucleic acid is 3′ to the second nucleic acid or the first nucleic acid is 5′ to the second nucleic acid). In some embodiments, the single polynucleotide is DNA. In some embodiments, the single polynucleotide is RNA. In select embodiments, the single polynucleotide is a mRNA.

Thus, the disclosure further provides a polynucleotide (e.g., an mRNA) comprising the first nucleic acid encoding a regulatory protein and the second nucleic acid encoding an immunogenic and/or antigenic polypeptide, or a single polynucleotide encoding the mRNA. In some embodiments, mRNA comprises a nucleic acid sequence encoding a viral regulatory protein and a nucleic acid encoding an immunogenic and/or antigenic viral polypeptide which has been codon-optimized to the codon usage of the virus from which the viral regulatory protein was derived.

The disclosure also provides polynucleotide segments encoding the mRNA, vectors containing these segments, and cells containing the vectors. The vectors may be used to propagate the segment in an appropriate cell and/or to allow expression from the segment (e.g., an expression vector). The person of ordinary skill in the art would be aware of the various vectors available for propagation and expression of a nucleic acid sequence.

The polynucleotide or mRNA may further comprise a sequence encoding a ribosome skipping peptide or internal ribosome entry site (IRES). This is particularly advantageous when a single nucleic acid or vector is used to express multiple components of the system. The ribosome skipping peptide may comprise a 2A family peptide. 2A peptides are short (˜-18-25 aa) peptides derived from viruses. There are four commonly used 2A peptides, P2A, T2A, E2A and F2A, that are derived from four different viruses. Any known 2A peptide sequence is suitable for use in the disclosed polynucleotide.

The single polynucleotide or mRNA may further comprise other structures and sequences necessary for proper functionality or stability, including but not limited to a 5′ untranslated region (UTR), a 5′ cap, a 3′ UTR, a 3′ tailing sequence, or any combination thereof.

5′-cap refers to the structure found on the 5′-end of an mRNA which ordinarily consists of a guanosine nucleotide connected to the mRNA via a 5′ to 5′ triphosphate linkage. The guanosine may be methylated at the 7-position creating a 7-methylguanosine cap (m⁷G). In some embodiments, the 5′ cap may be a naturally occurring 5′ cap. In some embodiments, the 5′ cap is a 5′ cap analog or a modified 5′ cap structure which is non-naturally occurring (e.g., phosphorothioate-cap-analogs).

The single polynucleotide or mRNA may comprise a 5′ and 3′ untranslated region (UTR). Sequence elements within the UTRs affect translational efficiency and RNA stability but do not encode a polypeptide. The 3′ UTR relates to the region located at the 3′ end of the mRNA, downstream of the termination codon of a protein-encoding region, which is transcribed but not translated into an amino acid sequence. The 5′UTR refers to the region directly upstream from the initiation codon. Eukaryotic 5′ UTRs contain the Kozak consensus sequence (ACCAUGG), which contains the initiation codon. The eukaryotic 5′ UTR may also contain cis-acting regulatory elements called upstream open reading frames (uORFs) and upstream AUGs (uAUGs) and termination codons, which can impact translation regulation.

The single polynucleotide or mRNA may comprise a 3′ tailing sequence. The 3′ tailing sequence comprises a polyA tail, a polyG quartet, a stem loop sequence, a triple helix forming sequence, a tRNA-like sequence, or any combination thereof.

In some embodiments, the single polynucleotide or mRNA further comprises a triple helix forming sequence. A triple helix is formed after the binding of a third strand to the major groove of a duplex nucleic acid through Hoogsteen base pairing (e.g., hydrogen bonds) while maintaining the duplex structure of two strands making the major groove. Pyrimidine-rich and purine-rich sequences (e.g., two pyrimidine tracts and one purine tract or vice versa) can form stable triplex structures as a consequence of the formation of triplets (e.g., A-U-A and C-G-C). In some embodiments, the triple helix sequence is derived from the 3′ terminal triple helix sequences of triple helix terminators from a long non-coding RNAs (lncRNAs), e.g., metastasis-associated lung adenocarcinoma transcript 1 (MALAT1).

In some embodiments, the single polynucleotide or mRNA further comprises a tRNA-like sequence. The tRNA-like sequences are those sequences which form similar overall secondary and tertiary structure to tRNA. In some embodiments, the tRNA-like sequence is derived from a long non-coding RNAs (lncRNAs), e.g., MALAT1. The tRNA-like sequence derived from lncRNAs may be truncated or modified as long as they retain the clover secondary structure.

As the MALAT1 sequences are highly conserved evolutionarily, the MALAT1 sequences for the triple helix or the tRNA-like sequence can be from any species. In one embodiment, the MALAT1 sequences are from a human. In another embodiment, the MALAT1 sequences are from a mouse. In another embodiment, the MALAT1 sequences are from a non-human primate.

The 3′ poly(A) sequence of mRNA is important for nuclear export, RNA stability and translational efficiency of eukaryotic messenger RNA (mRNA). The poly(A) tail is a segment of RNA at the 3′ end of the molecule that has only adenine bases. For example, a poly(A) tail may contain 10, 20, 30, 40, 50, 60, 70, 80, 90, 100, 110, 120, 130, 140, 150, 160, 170, 180, 190, 200, 210, 220, 230, 240, 250, 260, 270, 280, 290 or 300 adenosine monophosphates The poly(A) tail may contain two segments of only adenine bases separated by a linker.

In some embodiments, the single polynucleotide or mRNA comprises at least one chemical modification or chemically modified base or nucleoside. The chemical modifications may comprise any modification which is not naturally present in said RNA or any naturally-occurring modification of adenosine (A), guanosine (G), uridine (U), or cytidine (C) ribonucleosides. For example, a single mRNA may include both naturally-occurring and non-naturally-occurring modifications. Chemical modifications may be located in any portion of the mRNA molecule and the mRNA molecule may contain any percentage of modified nucleosides (1-100%). In some embodiments, every particular base or nucleoside may be modified (e.g., every uridine is a modified uridine). In some embodiments, a particular modification is used for every particular type of nucleoside or base (e.g., every uridine is modified to a 1-methyl-pseudouridine). Exemplary RNA modifications can be found in the RNA modification database (See, mods.rna.albany.edu/home).

In some embodiments, the at least one chemical modification comprises a modified uridine residue. Exemplary modified uridine residues include, but are not limited to, pseudouridine, 1-methylpseudouridine, 1-ethylpseudouridine, 2-thiouridine, 4′-thiouridine, 5-methyluridine, 2-thio-1-methyl-1-deaza-pseudouridine, 2-thio-1-methyl-pseudouridine, 2-thio-5-aza-uridine, 2-thio-dihydropseudouridine, 2-thio-dihydrouridine, 2-thio-pseudouridine, 4-methoxy-2-thio-pseudouridine, 4-methoxy-pseudouridine, 4-thio-1-methyl-pseudouridine, 4-thio-pseudouridine, 5-aza-uridine, dihydropseudouridine, 5-methoxyuridine and 2′-O-methyl uridine.

In some embodiments, the at least one chemical modification comprises a modified cytosine residue. Exemplary nucleosides having a modified cytosine include 5-aza-cytidine, 6-aza-cytidine, pseudoisocytidine, 3-methyl-cytidine, N4-acetyl-cytidine, 5-formyl-cytidine, N4-methyl-cytidine, 5-methyl-cytidine, 5-halo-cytidine, 5-hydroxymethyl-cytidine, 1-methyl-pseudoisocytidine, pyrrolo-cytidine, pyrrolo-pseudoisocytidine, 2-thio-cytidine, 2-thio-5-methyl-cytidine, 4-thio-pseudoisocytidine, 4-thio-1-methyl-pseudoisocytidine, 4-thio-1-methyl-1-deaza-pseudoisocytidine, 1-methyl-1-deaza-pseudoisocytidine, zebularine, 5-aza-zebularine, 5-methyl-zebularine, 5-aza-2-thio-zebularine, 2-thio-zebularine, 2-methoxy-cytidine, 2-methoxy-5-methyl-cytidine, 4-methoxy-pseudoisocytidine, 4-methoxy-1-methyl-pseudoisocytidine, lysidine, α-thio-cytidine, 2′-O-methyl-cytidine, 5,2′-O-dimethyl-cytidine, N4-acetyl-2′-O-methyl-cytidine. N4,2′-O-dimethyl-cytidine, 5-formyl-2′-O-methyl-cytidine, N4,N4,2′-O-trimethyl-cytidine, 1-thio-cytidine, 2′-F-aracytidine, 2′-F-cytidine, and 2′-OH-aracytidine.

In some embodiments, the at least one chemical modification comprises a modified adenine residue. Exemplary nucleosides having a modified adenine include 2-amino-purine, 2,6-diaminopurine, 2-amino-6-halo-purine, 6-halo-purine, 2-amino-6-methyl-purine, 8-azido-adenosine, 7-deaza-adenine, 7-deaza-8-aza-adenine, 7-deaza-2-amino-purine, 7-deaza-8-aza-2-amino-purine, 7-deaza-2,6-diaminopurine, 7-deaza-8-aza-2,6-diaminopurine, 1-methyl-adenosine, 2-methyl-adenine, N6-methyl-adenosine, 2-methylthio-N6-methyl-adenosine, N6-isopentenyl-adenosine, 2-methylthio-N6-isopentenyl-adenosine, N6-(cis-hydroxyisopentenyl)adenosine, 2-methylthio-N6-(cis-hydroxyisopentenyl)adenosine, N6-glycinylcarbamoyl-adenosine. N6-threonylcarbamoyl-adenosine, N6-methyl-N6-threonylcarbamoyl-adenosine, 2-methylthio-N6-threonylcarbamoyl-adenosine, N6,N6-dimethyl-adenosine, N6-hydroxynorvalylcarbamoyl-adenosine, 2-methylthio-N6-hydroxynorvalylcarbamoyl-adenosine, N6-acetyl-adenosine, 7-methyl-adenine, 2-methylthio-adenine, 2-methoxy-adenine. α-thio-adenosine, 2′-O-methyl-adenosine, N6,2′-O-dimethyl-adenosine, N6,N6,2′-O-trimethyl-adenosine, 1,2′-O-dimethyl-adenosine, 2′-O-ribosyladenosine (phosphate), 2-amino-N6-methyl-purine, 1-thio-adenosine, 8-azido-adenosine, 2′-F-ara-adenosine, 2′-F-adenosine, 2′-OH-ara-adenosine, and N6-(19-amino-pentaoxanonadecyl)-adenosine.

In some embodiments, the at least one chemical modification comprises a modified guanine residue. Exemplary nucleosides having a modified guanine include inosine, 1-methyl-inosine, wyosine, methylwyosine, 4-demethyl-wyosine, isowyosine, wybutosine, peroxywybutosine, hydroxywybutosine, undermodified hydroxywybutosine, 7-deaza-guanosine, queuosine, epoxyqueuosine, galactosyl-queuosine, mannosyl-queuosine, 7-cyano-7-deaza-guanosine, 7-aminomethyl-7-deaza-guanosine, archaeosine, 7-deaza-8-aza-guanosine, 6-thio-guanosine, 6-thio-7-deaza-guanosine, 6-thio-7-deaza-8-aza-guanosine, 7-methyl-guanosine, 6-thio-7-methyl-guanosine, 7-methyl-inosine, 6-methoxy-guanosine, 1-methyl-guanosine, N2-methyl-guanosine, N2,N2-dimethyl-guanosine, N2,7-dimethyl-guanosine, N2,N2,7-dimethyl-guanosine, 8-oxo-guanosine, 7-methyl-8-oxo-guanosine, 1-methyl-6-thio-guanosine, N2-methyl-6-thio-guanosine, N2,N2-dimethyl-6-thio-guanosine, α-thio-guanosine, 2′-O-methyl-guanosine, N2-methyl-2′-O-methyl-guanosine, N2,N2-dimethyl-2′-O-methyl-guanosine, 1-methyl-2′-O-methyl-guanosine, N2,7-dimethyl-2′-O-methyl-guanosine, 2′-O-methyl-inosine, 1,2′-O-dimethyl-inosine, and 2′-O-ribosylguanosine (phosphate).

The compositions may further comprise excipients or pharmaceutically acceptable carriers. The choice of excipients or pharmaceutically acceptable carriers will depend on factors including, but not limited to, the particular mode of administration, the effect of the excipient on solubility and stability, and the nature of the dosage form.

Excipients and carriers may include any and all solvents, dispersion media, coatings, antibacterial and antifungal agents, isotonic and absorption delaying agents. Some examples of materials which can serve as excipients and/or carriers are sugars including, but not limited to, lactose, glucose and sucrose, starches including, but not limited to, corn starch and potato starch; cellulose and its derivatives including, but not limited to, sodium carboxymethyl cellulose, ethyl cellulose and cellulose acetate; powdered tragacanth: malt; gelatin; talc: excipients including, but not limited to, cocoa butter and suppository waxes; oils including, but not limited to, peanut oil, cottonseed oil, safflower oil, sesame oil, olive oil, corn oil and soybean oil; glycols; including propylene glycol; esters including, but not limited to, ethyl oleate and ethyl laurate: agar; buffering agents including, but not limited to, magnesium hydroxide and aluminum hydroxide; alginic acid; pyrogen-free water; isotonic saline; Ringer's solution; ethyl alcohol, and phosphate buffer solutions, as well as other non-toxic compatible lubricants including, but not limited to, sodium lauryl sulfate and magnesium stearate, as well as coloring agents, releasing agents, coating agents, sweetening, flavoring and perfuming agents, preservatives and antioxidants. The compositions of the present invention and methods for their preparation will be readily apparent to those skilled in the art. Techniques and formulations may be found, for example, in Remington's Pharmaceutical Sciences, 19th Edition (Mack Publishing Company, 1995).

The compositions may be formulated for any particular mode of administration including for example, systemic administration (e.g., oral, rectal, nasal, sublingual, buccal, implants, or parenteral).

The compositions may further comprise a delivery vehicle. Exemplary delivery vehicles include, but are not limited to, microparticle compositions comprising poly(lactic acid) (PLA) and/or poly(lactic-co-glycolic acid) (PLGA), albumin nanoparticles, and liposomal compositions.

The compositions may further comprise a lipid nanoparticle encapsulating the disclosed polynucleotides or mRNA using well known technology.

Lipid nanoparticle compositions of the disclosure may include one or more cationic and/or ionizable lipids, phospholipids, neutral or non-cationic lipids, polyethylene glycol (PEG)-lipid conjugates, and/or sterols. In some embodiments, the lipid nanoparticle comprises a cationic lipid and/or ionizable lipid, a neutral or non-cationic lipid, and cholesterol.

Cationic and/or ionizable lipids include, for example, amine-containing lipids that can be readily protonated and may have a positive or partial positive charge at physiological pH due to a pKa value between pH 5 and 8. The polar headgroup of the cationic lipids preferably comprises amine derivatives such as primary, secondary, and/or tertiary amines, quaternary ammonium, various combinations of amines, amidinium salts, or guanidine and/or imidazole groups as well as pyridinium, piperazine and amino acid headgroups such as lysine, arginine, ornithine and/or tryptophan. Cationic lipids include, but are not limited to, 1,2-dimyristoyl-sn-glycero-3-ethylphosphocholine (DMEPC), 1,2-di-O-octadecenyl-3-trimethylammonium propane (DOTMA) and/or 1,2-dioleoyl-3-trimethylammonium propane (DOTAP), 1,2-dimyristoyl-3-trimethylammonium propane (DMTAP), 2,3-di(tetradeoxy)propyl-(2-hydroxyethyl)-dimethylazanium bromide (DMRIE), didodecyl(dimethyl)azanium bromide (DDAB), 1,2-dioleyloxypropyl-3-dimethyl-hydroxyethyl ammonium bromide (DORIE), 3β-[N—(N\N′-dimethylamino-ethane)carbamoyl]cholesterol (DC-Chol) or dioleyl ether phosphatidylcholine (DOEPC). Ionizable lipids include, but are not limited to, 1,2-dioleyloxy-3-dimethylamino-propane (DODMA).

In some embodiments, the lipid nanoparticle comprises a polyethylene glycol (PEG)-lipid conjugate. A PEG-lipid conjugate may include, but is not limited to, PEG-modified phosphatidylethanolamines, PEG-modified phosphatidic acids, PEG-modified ceramides, PEG-modified dialkylamines, PEG-modified diacylglycerols, PEG-modified dialkylglycerols, and mixtures thereof. For example, a PEG lipid may be PEG-DMG (1,2-dimyristoyl-rac-glycero-3-methoxypolyethylene glycol), PEG-c-DOMG (R-3-[(ω-methoxy poly(ethylene glycol)2000)carbamoyl)]-1,2-dimyristyloxlpropyl-3-amine), PEG-DMA (PEG-dimethacrylate), PEG-DLPE (1,2-didodecanoyl-sn-glycero-3-phosphoethanolamine-PEG), PEG-DMPE (PEG-1,2-dimyristoyl-sn-glycero-3-phosphoethanolamine), PEG-DPPC (PEG-dipalmitoyl phosphatidylcholine), PEG-N,N-di(tetradecyl)acetamide, or a PEG-DSPE (1,2-distearoyl-sn-glycero-3-phosphoethanolamine-poly(ethylene glycol)) lipid. In some embodiments, the lipid nanoparticle comprises PEG-DMG and/or PEG-N,N-di(tetradecyl)acetamide.

The sterol may comprise cholesterol, fecosterol, ergosterol, campesterol, sitosterol, stigmasterol, brassicasterol or a sterol ester, such as cholesteryl hemisuccinate, cholesteryl sulfate, or any other derivatives of cholesterol.

A neutral or non-cationic lipid may include one or more phospholipids. Phospholipids include a phospholipid moiety and one or more fatty acid moieties. A phospholipid moiety may include, but is not limited to, phosphatidyl choline, phosphatidyl ethanolamine, phosphatidyl glycerol, phosphatidyl serine, phosphatidic acid, 2-lysophosphatidyl choline, and sphingomyelin. A fatty acid moiety may include, but is not limited to, lauric acid, myristic acid, myristoleic acid, palmitic acid, palmitoleic acid, stearic acid, oleic acid, linoleic acid, alpha-linolenic acid, erucic acid, phytanic acid, arachidic acid, arachidonic acid, eicosapentanoic acid, behenic acid, docosapentaenoic acid, and docosahexaenoic acid.

Phospholipids suitable for use in the compositions may include, but are not limited to, phosphatidylglycerol (PG) including dimyristoyl phosphatidylglycerol (DMPG) and 1,2-dioleoyl-sn-glycero-3-phospho-rac-(I-glycerol) sodium salt (DOPG); phosphatidylcholine (PC), including egg yolk phosphatidylcholine, dimyristoyl phosphatidylcholine (DMPC), 1,2-distearoyl-sn-glycero-3-phosphocholine (DSPC), 1,2-dilinolenoyl-sn-glycero-3-phosphocholine (DLPC), 1,2-dioleoyl-sn-glycero-3-phosphocholine (DOPC), 1,2-dipalmitoyl-sn-glycero-3-phosphocholine (DPPC), 1,2-diundecanoyl-sn-glycero-phosphocholine (DUPC), 1-palmitoyl-2-oleoyl-sn-glycero-3-phosphocholine (POPC), 1,2-di-O-octadecenyl-sn-glycero-3-phosphocholine (18:0 Diether PC), 1-oleoyl-2-cholesterylhemisuccinoyl-sn-glycero-3-phosphocholine (OChemsPC), 1-hexadecyl-sn-glycero-3-phosphocholine (C16 Lyso PC), 1,2-dilinolenoyl-sn-glycero-3-phosphocholine, 1,2-didocosahexaenoyl-sn-glycero-3-phosphocholine, 1,2-diarachidonoyl-sn-glycero-3-phosphocholine; phosphatidylethanolamine (PE) including 1,2-dioleoyl-sn-glycero-3-phosphoethanolamine (DOPE), 1,2-diphytanoyl-sn-glycero-3-phosphoethanolamine (ME 16.0 PE), 1,2-distearoyl-sn-glycero-3-phosphoethanolamine, 1,2-dilinolenoyl-sn-glycero-3-phosphoethanolamine, 1,2-dilinolenoyl-sn-glycero-3-phosphoethanolamine, 1,2-diarachidonoyl-sn-glycero-3-phosphoethanolamine, 1,2-didocosahexaenoyl-sn-glycero-3-phosphoethanolamine; phosphatidic acid (PA); phosphatidylinositol (PI); phosphatidylserine (PS); and sphingomyelin (SM). In some embodiments, the lipid nanoparticle comprises DSPC.

The positively charged lipid structures described herein may also include other components typically used in the formation of vesicles (e.g., for stabilization). Examples of such other components includes, without being limited thereto, fatty alcohols, fatty acids, and/or any other pharmaceutically acceptable excipients which may affect the surface charge, the membrane fluidity and assist in the incorporation of the lipid into the lipid assembly.

### 3. Vaccines or Medicaments

The compositions, polynucleotides, or mRNA described herein may be used to prepare vaccines or another medicament.

The vaccine or medicament may comprise an adjuvant or immunostimulant, or a polynucleotide encoding an adjuvant or immunostimulant (e.g., an adjuvantive polypeptide). Adjuvants and immunostimulants are compounds or compositions that either directly or indirectly stimulate the immune system's response to a co-administered antigen. In some embodiments, the mRNA-vaccines are not adjuvanted or are self-adjuvanting.

Suitable adjuvants are commercially available as, for example, Glucopyranosyl Lipid Adjuvant (GLA); Pam3CSK4; Freund's Incomplete Adjuvant and Complete Adjuvant (Difco Laboratories. Detroit. Mich.); Merck Adjuvant 65 (Merck and Company, Inc., Rahway, N.J.); AS-2 (SmithKline Beecham); mineral salts (for example, aluminum, silica, kaolin, and carbon); aluminum salts such as aluminum hydroxide gel (alum). AlK(SO₄)₂, AlNa(SO₄)₂, AlNH₄(SO₄), and Al(OH)₃; salts of calcium (e.g., Ca₃(PO₄)₂), iron or zinc: an insoluble suspension of acylated tyrosine; acylated sugars; cationically or anionically derivatized polysaccharides; polynucleotides (for example, poly IC, poly AU acids, and CpG oligodeoxynucleotides (e.g., Class A or B)): polyphosphazenes; cyanoacrylates; polymerase-(DL-lactide-co-glycoside); bovine serum albumin; diphtheria toxoid; tetanus toxoid; edestin; keyhole-limpet hemocyanin; Pseudomonil Toxin A: choleragenoid; cholera toxin; pertussis toxin, viral proteins; Quil A; aminoalkyl glucosamine phosphate compounds. In addition, adjuvants such as cytokines (e.g., GM-CSF or interleukin-2, -7, or -12), interferons, or tumor necrosis factor, may also be used as adjuvants. Protein and polypeptide adjuvants may be obtained from natural or recombinant sources according to methods well known to those skilled in the art. When obtained from recombinant sources, the adjuvant may comprise a protein fragment comprising at least the immunostimulatory portion of the molecule.

Other known immunostimulatory macromolecules which can be used include, but are not limited to, polysaccharides, tRNA, non-metabolizable synthetic polymers such as polyvinylamine, polymethacrylic acid, polyvinylpyrrolidone, mixed polycondensates (with relatively high molecular weight) of 4′,4-diaminodiphenylmethane-3,3′-dicarboxylic acid and 4-nitro-2-aminobenzoic acid (See, Sela, M., Science 166: 1365-1374 (1969)) or glycolipids, lipids, or carbohydrates.

In some embodiments, the adjuvantive polypeptide comprises immune activator proteins, such as CD70, CD40 ligand, and constitutively active TLR4, or polycationic peptides (e.g., protamine). In some embodiments, the adjuvantive polypeptide is a flagellin polypeptide. Commercially available mRNA encoding adjuvantive polypeptides are available, for example, as TriMix (See Bonehill, A. et al. Mol. Ther. 16, 1170-1180 (2008), incorporated herein by reference).

The vaccine or medicament of the present disclosure may also contain other compounds, which may be biologically active or inactive. For example, one or more immunogenic and antigenic portions of other polypeptides or polynucleotides encoding immunogenic polypeptides may be present within the vaccine. The vaccine or medicament may generally be used for prophylactic and therapeutic purposes.

The vaccines or medicaments may be formulated for any appropriate manner of administration, and thus administered, including for example, topical, oral, nasal, intravenous, intravaginal, epicutaneous, sublingual, intracranial, intradermal, intraperitoneal, subcutaneous, intramuscular administration, or via inhalation.

The vaccines or medicaments may also comprise buffers (e.g., neutral buffered saline or phosphate buffered saline), carbohydrates (e.g., glucose, mannose, sucrose or dextrans), mannitol, proteins, polypeptides, or amino acids such as glycine, antioxidants, bacteriostats, chelating agents such as EDTA or glutathione, solutes that render the formulation isotonic, hypotonic, or weakly hypertonic with the blood of a recipient, suspending agents, thickening agents and/or preservatives. Alternatively, vaccines or medicaments may be formulated as a lyophilisate.

### 4. Methods

The present disclosure provides methods for reducing treating, reducing, or preventing a disease or disorder (e.g., an infection) in a subject in need thereof. The present disclosure also provides methods of inducing an immune response in a subject. The methods include administering to the subject an effective amount of the compositions or vaccines or medicaments disclosed herein.

An “effective amount” is an amount that is delivered to a subject, either in a single dose or as part of a series, which is effective for inducing a response in the subject. This amount varies depending upon the health and physical condition of the subject to be treated, the capacity of the subject's immune system to synthesize antibodies, the formulation of the compositions, and other relevant factors. It is expected that the amount will fall in a relatively broad range that can be determined by one of skill in the art through routine trials.

The viral infection may be a coronavirus infection. In some embodiments, the coronavirus is severe acute respiratory syndrome coronavirus-2 (SARS-CoV-2).

The compositions, vaccines, or medicaments disclosed herein can be administered in a wide variety of therapeutic dosage forms. The route and regimen of administration will vary depending upon the population and the indication for vaccination and is to be determined by the skilled practitioner. For example, the compositions, vaccines, or medicaments disclosed herein may be administered parentally, e.g., in intravenous (either by bolus or infusion methods), intraperitoneal, subcutaneous, topical with or without occlusion, or intramuscular form.

The administration may comprise an initial immunization or dose and at least one subsequent immunization or booster dose, following known standard immunization protocols. The boosting doses will be adequately spaced at such times where the levels of circulating antibody fall below a desired level. Boosting doses may consist of the compositions or vaccines disclosed herein and may comprise alternative carriers and/or adjuvants. The booster dosage levels may be the same or different that those of the initial immunization dosage.

The specific dose level may depend upon a variety of factors including the activity of the polynucleotides (e.g., mRNA), composition, vaccine, or medicament, the age, body weight, general health, and diet of the subject, time of administration, and route of administration. For prophylaxis purposes, the amount of the polynucleotide(s) (e.g., mRNA) in each dose is an amount which induces an immunoprotective response without significant adverse side effects.

The compositions and vaccines may be prepared, packaged, or sold in a form suitable for bolus administration or sold in unit dosage forms, such as in ampules or multi-dose containers. In some embodiments, the compositions and vaccines contain a preservative. Also disclosed herein is a system or kit comprising the compositions, polynucleotides, or vaccines disclosed herein and a delivery device or container. In some embodiments, the delivery device or container comprises a syringe or syringe vial. In some embodiments, the delivery device or container is pre-filled with the composition.

### 5. EXAMPLES

The following are examples of the present invention and are not to be construed as limiting.

### Example 1

Codon usage was analyzed using the graphical codon usage analyzer (GCUA) software (gcua.schoedl.de) and Prism9 software. Codon-optimized sequences were synthesized in vitro (GenScript).

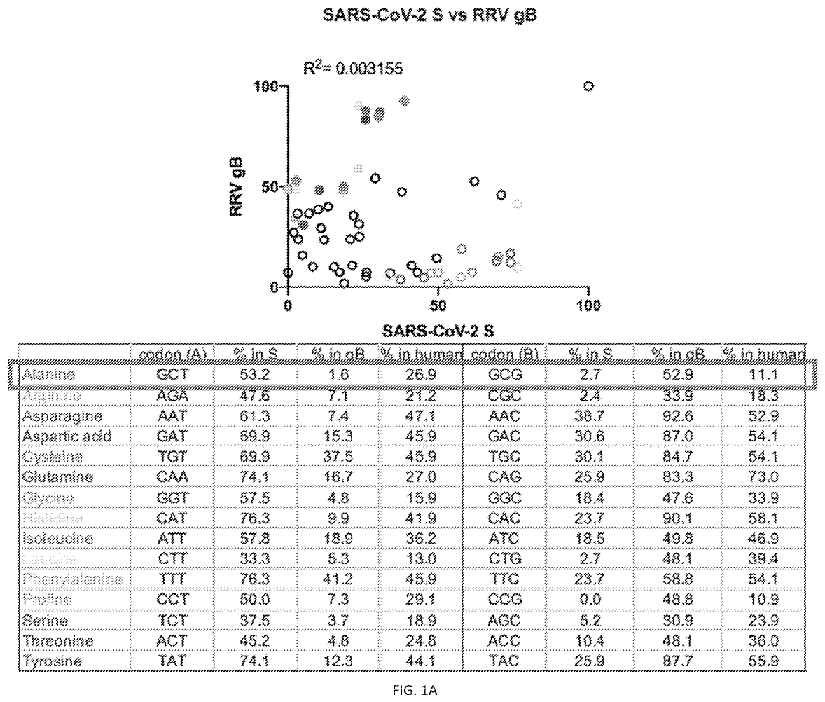

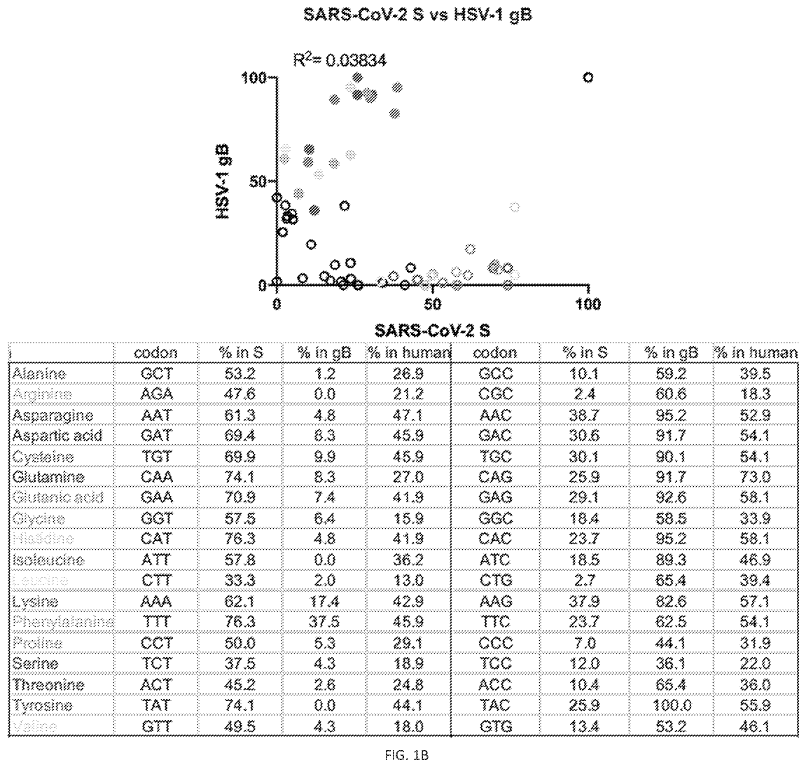

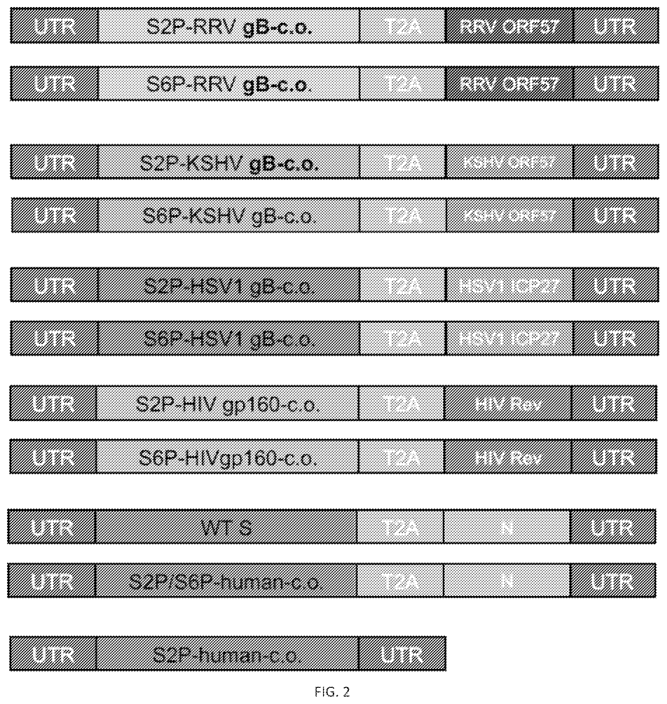

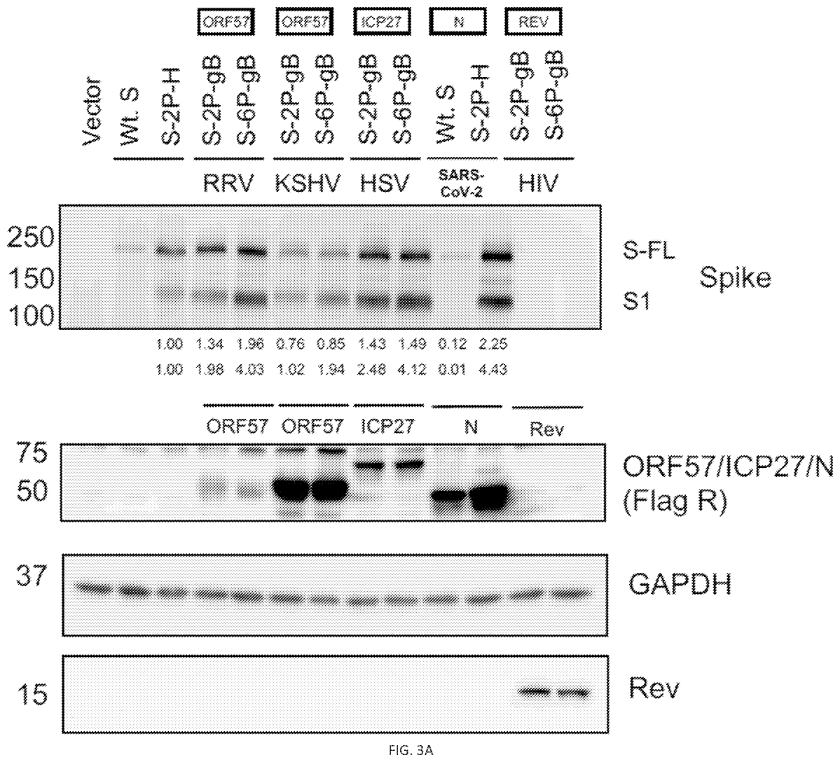

FIG. 2 shows exemplary constructs utilizing SARS COV-2 spike proteins (S2P, S6P and WT S) in a single mRNA with a corresponding regulatory protein. In some instances, the spike proteins were codon optimized (c.o.) to the virus from which the corresponding regulatory protein was derived, as indicated. A comparison of codon usage and exemplar schematic for codon optimization is shown in FIGS. 1A and 1B for SARS-CoV-2 spike protein with RRV glycoprotein B or HSV glycoprotein B, respectively.

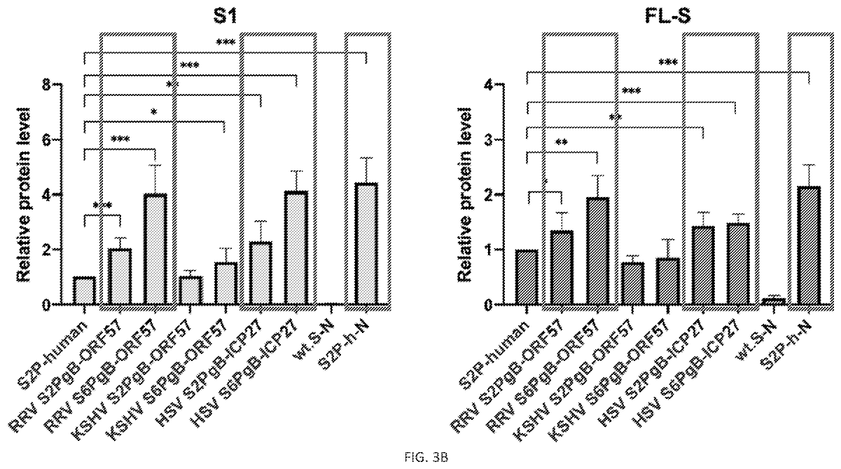

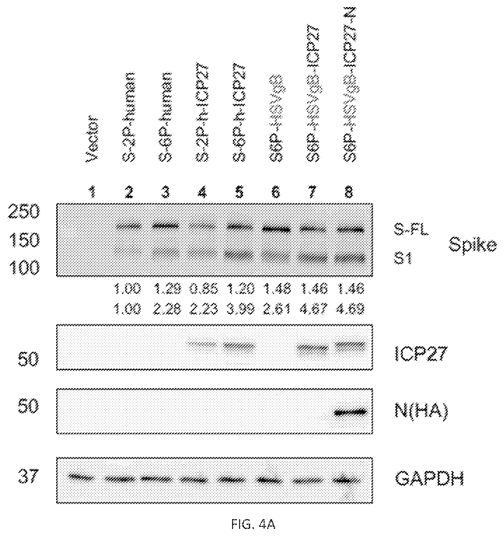

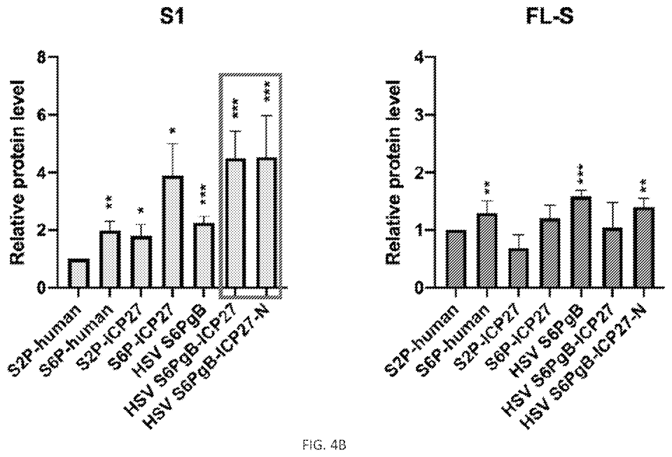

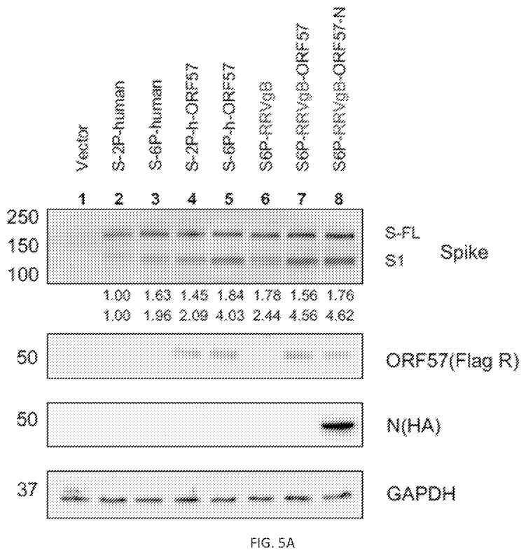

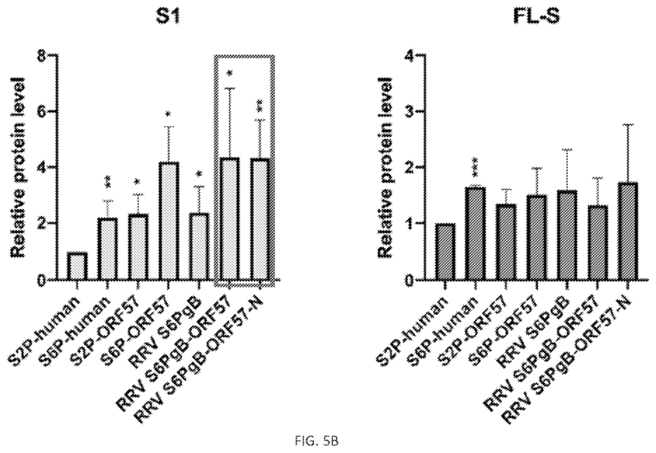

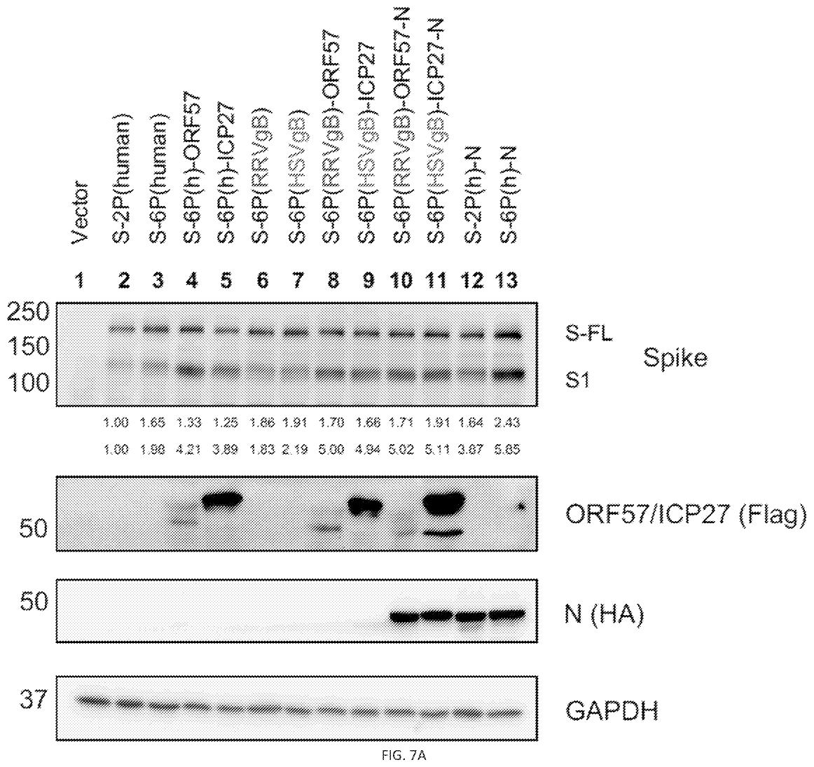

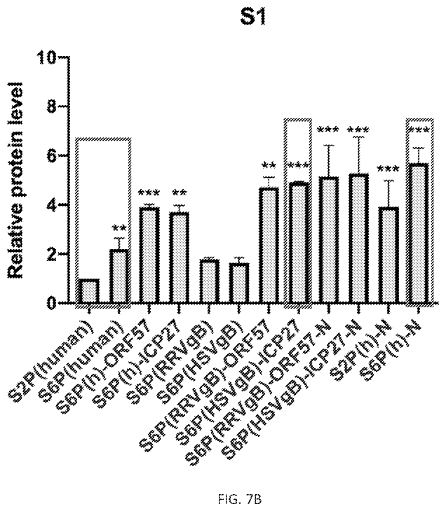

As shown in FIG. 3, expression of SARS CoV-2 spike protein S2P was increased when co-expressed with a regulatory protein (See boxed results in FIG. 3B). S2P or S6P can be upregulated in the presence of ICP27, ORF57, or SARS CoV-2 N (FIGS. 4-5 and 7). gB codon optimization on S6P showed a similar pattern with human codon optimization. Moreover, the presence of ICP27 or ORF57 mainly boosts the S1 level based on gB codon optimization.

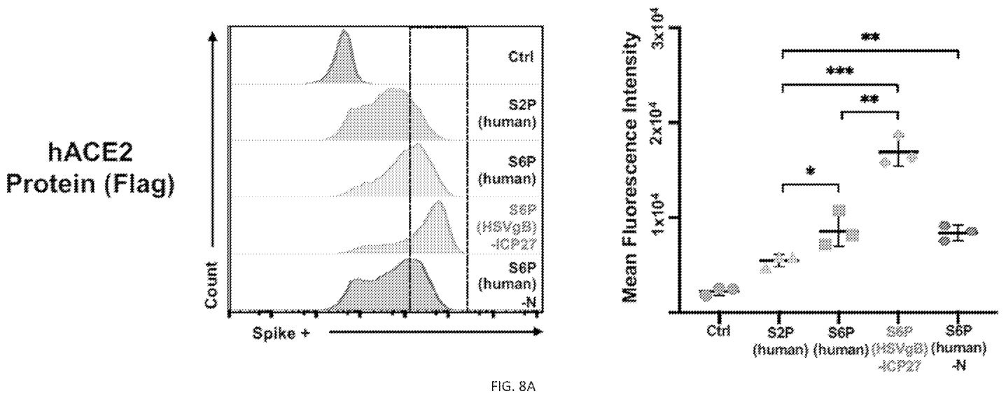

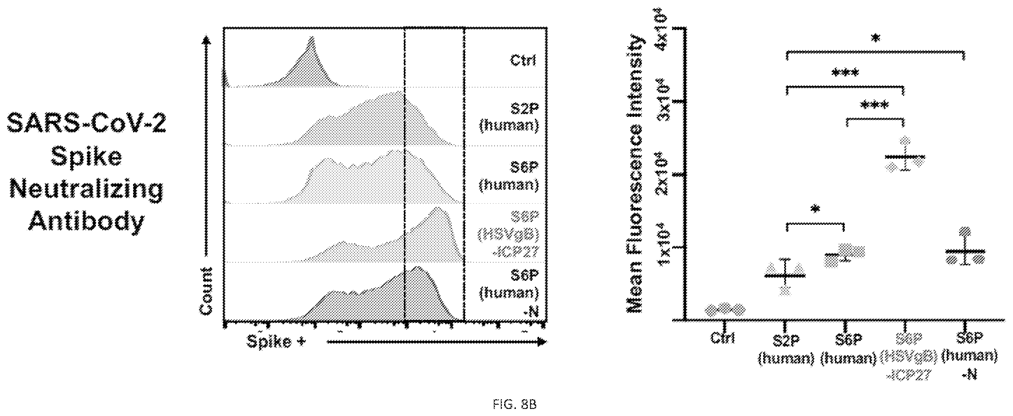

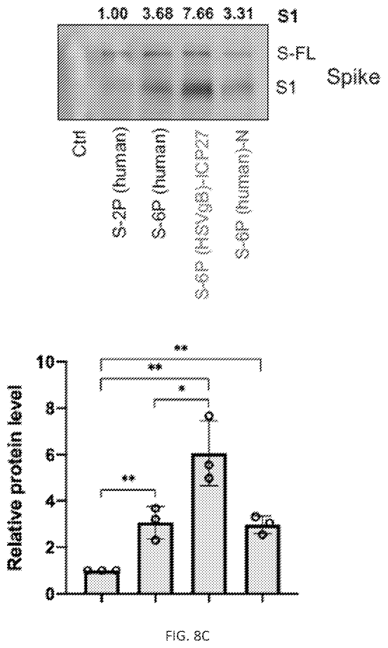

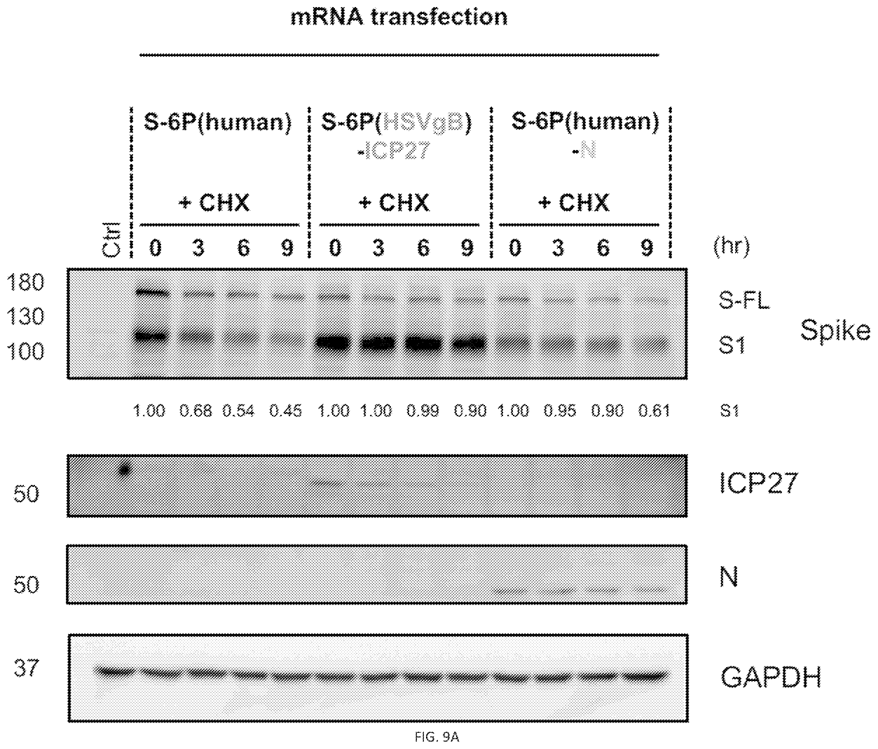

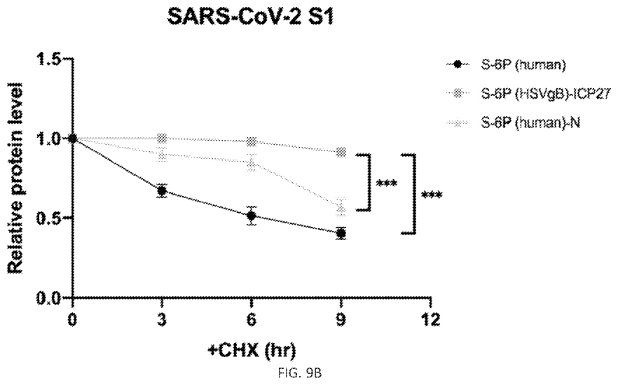

S2P, S6p, S6P(HSVgB)—ICP27, and S6P(h)—N constructs are all less than ˜5.5 kb and facilitate encapsulation of mRNA. The spike surface expression for each of these constructs was determined following mRNA transfection (FIG. 8). S6P(HSVgB)—ICP27 showed the highest surface expression, which was highly indicative of correct protein folding. Protein stability was studied using a cycloheximide chase assay. Based on the results shown in FIG. 9, ICP27 or SARS-CoV-2 N may confer an increased stability of Spike protein expression, particularly ICP27, thus decreasing the chance of inefficiencies due to protein degradation.

### Example 2

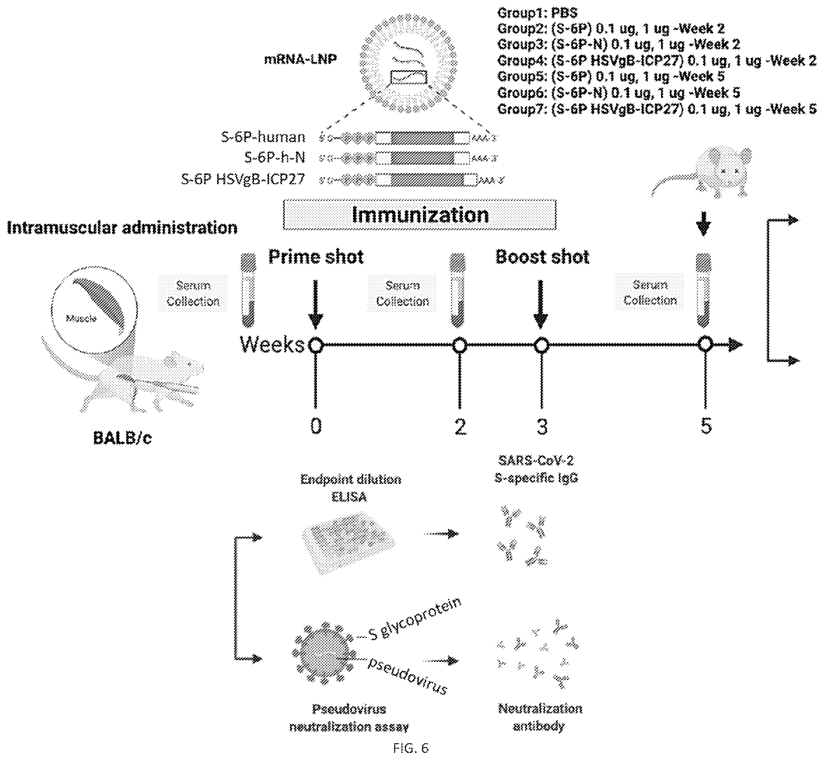

FIG. 6 shows a schematic of an exemplary method for determining the humoral response induced by the exemplary constructs utilizing SARS COV-2 spike proteins (S2P, S6P, S-6P HSVgB-ICP27, and S-6P—N) in a single mRNA. Specifically, the constructs are assembled into lipid nanoparticles (LNP) for primary and booster immunizations. After a period of time, blood samples are collected and analyzed for the presence of SARS-CoV-2 spike protein specific antibodies.

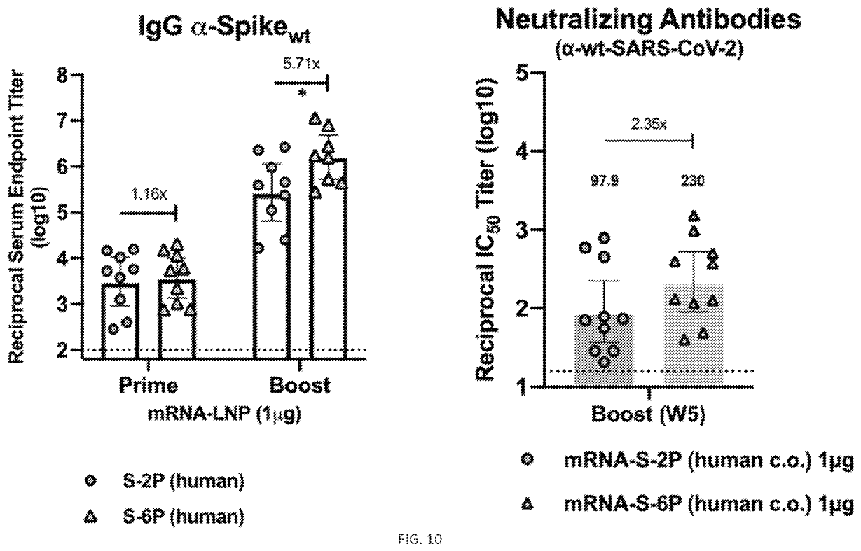

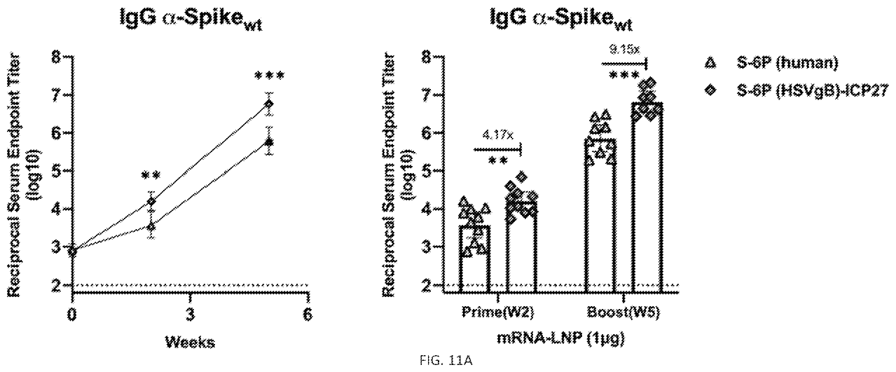

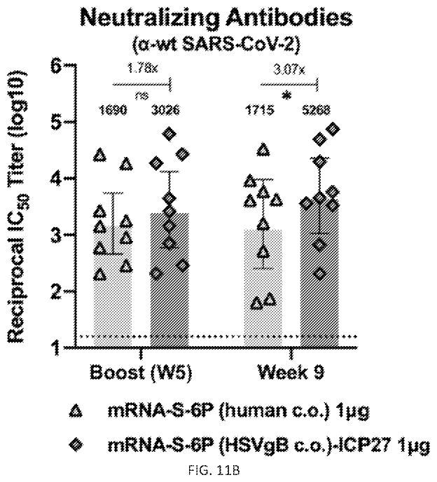

As shown in FIG. 10, the level of IgG or neutralizing antibodies from mouse serum against wild-type Spike protein can be determined by endpoint ELISA or pseudovirus neutralization assays. S-6P mRNA-LNP stimulated higher levels of IgG or neutralizing antibodies compared to S-2P mRNA LNP. HSVgB codon optimized S-6P with ICP27 mRNA-LNP stimulated higher levels of IgG or neutralizing antibodies than S-6P human codon-optimized mRNA-LNP (FIG. 11).

The scope of the present invention is not limited by what has been specifically shown and described hereinabove. Those skilled in the art will recognize that there are suitable alternatives to the depicted examples of materials, configurations, constructions, and dimensions. Variations, modifications, and other implementations of what is described herein will occur to those of ordinary skill in the art without departing from the spirit and scope of the invention.

Numerous references, including patents and various publications, are cited and discussed in the description of this invention. The citation and discussion of such references is provided merely to clarify the description of the present invention and is not an admission that any reference is prior art to the invention described herein. All references cited and discussed in this specification are incorporated herein by reference in their entirety.

What is claimed is:

1. A composition comprising:
a first nucleic acid encoding a viral regulatory protein; and
a second nucleic acid encoding an immunogenic polypeptide,
wherein the immunogenic polypeptide is codon-optimized to a virus from which the regulatory protein is derived.

2. The composition of claim 1, wherein the regulatory protein comprises an immediate-early viral protein.

3. The composition of claim 1 or 2, wherein the regulatory protein comprises an ICP27 protein.

4. The composition of claim 3, wherein the ICP27 protein is derived from herpes simplex virus (HSV).

5. The composition of claim 1 or 2, wherein the regulatory protein comprises an ORF57 protein.

6. The composition of claim 5, wherein the ORF57 protein is derived from Kaposi's sarcoma-associated herpesvirus (KSHV) or rhesus monkey rhadinovirus (RRV).

7. The composition of any of claims 1-6, wherein the immunogenic polypeptide is derived from a pathogen or infectious agent.

8. The composition of claims 1-7, wherein the immunogenic polypeptide comprises a viral structural protein.

9. The composition of any of claims 1-8, wherein the immunogenic polypeptide comprises a viral spike protein or a fragment or variant thereof.

10. The composition of claim 9, wherein the viral spike protein or a fragment or variant thereof is from a coronavirus.

11. The composition of claim 10, wherein the coronavirus is SARS-CoV-2.

12. The composition of any of claims 7-11, wherein the regulatory protein and the immunogenic polypeptide are derived from different viruses.

13. The composition of claim 12, wherein the regulatory protein comprises HSV ICP27 protein and the viral immunogenic polypeptide comprises SARS-CoV-2 spike protein or a fragment or variant thereof codon-optimized to HSV glycoprotein B.

14. The composition of claim 12, wherein the regulatory protein comprises RRV ORF57 protein and the viral immunogenic polypeptide comprises SARS-CoV-2 spike protein or a fragment or variant thereof codon-optimized to RRV glycoprotein B.

15. The composition of any of claims 1-14, wherein a single polynucleotide comprises the first nucleic acid and the second nucleic acid.

16. The composition of claim 15, wherein the single polynucleotide is an mRNA.

17. The composition of claim 15 or 16, wherein the single polynucleotide further comprises a sequence encoding a ribosome skipping peptide.

18. The composition of claim 17, wherein the ribosome skipping peptide comprises a 2A family peptide.

19. The composition of any of claims 15-18, wherein the single polynucleotide further comprises or encodes: a 5′ untranslated region (UTR), a 5′ cap, a 3′ UTR, a 3′ tailing sequence or any combination thereof.

20. The composition of claim 19, wherein the 3′ tailing sequence comprises a polyA tail, a polyG quartet, a stem loop sequence, a triple helix forming sequence, a tRNA-like sequence, or any combination thereof.

21. A polynucleotide comprising:
a first nucleic acid sequence encoding a viral regulatory protein; and
a second nucleic acid sequence encoding an immunogenic polypeptide,
wherein the immunogenic polypeptide is codon-optimized to a virus from which the regulatory protein is derived.

22. The polynucleotide of claim 21, wherein the regulatory protein comprises an immediate-early viral protein.

23. The polynucleotide of claim 21 or 22, wherein the regulatory protein comprises an ICP27 protein.

24. The polynucleotide of claim 23, wherein the ICP27 protein is derived from HSV.

25. The polynucleotide of claim 21 or 22, wherein the regulatory protein comprises an ORF57 protein.

26. The polynucleotide of claim 25, wherein the ORF57 protein is derived from KSHV or RRV.

27. The polynucleotide of any of claims 21-26, wherein the immunogenic polypeptide is derived from a pathogen or infectious agent.

28. The polynucleotide of any of claims 21-27, wherein the immunogenic polypeptide comprises a viral structural protein.

29. The polynucleotide of any of claims 21-28, wherein the immunogenic polypeptide comprises a viral spike protein or a fragment or variant thereof.

30. The polynucleotide of claim 29, wherein the viral spike protein or a fragment or variant thereof is from a coronavirus.

31. The polynucleotide of claim 30, wherein the coronavirus is SARS-CoV-2.

32. The polynucleotide of any of claims 28-31, wherein the regulatory protein and the immunogenic polypeptide are derived from different viruses.

33. The polynucleotide of claim 32, wherein the regulatory protein comprises HSV ICP27 and the viral immunogenic polypeptide comprises SARS-CoV-2 spike protein or a fragment or variant thereof codon-optimized to HSV glycoprotein B.

34. The polynucleotide of claim 32, wherein the regulatory protein comprises RRV ORF57 and the viral immunogenic polypeptide comprises SARS-CoV-2 spike protein or a fragment or variant thereof codon-optimized to RRV glycoprotein B.

35. The polynucleotide of any of claims 21-34, wherein the polynucleotide is an mRNA.

36. The polynucleotide of any of claims 21-35, further comprising a ribosome skipping peptide sequence or a sequence encoding thereof.

37. The polynucleotide of claim 36, wherein the ribosome skipping peptide comprises a 2A family peptide.

38. The polynucleotide of any of claims 21-37, further comprising: a 5′ untranslated region (UTR), a 5′ cap, a 3′ UTR, a 3′ tailing sequence or any combination thereof.

39. The polynucleotide of claim 38, wherein the 3′ tailing sequence comprises a polyA tail, a polyG quartet, a stem loop sequence, a triple helix forming sequence, a tRNA-like sequence, or any combination thereof.

40. A vaccine comprising:
the composition of any of claims 1-20 or the polynucleotide of any of claims 21-39; and
at least one adjuvant, a delivery vehicle, or a combination thereof.

41. The vaccine of claim 40, wherein the delivery vehicle comprises a lipid nanoparticle encapsulating the composition or polynucleotide.

42. The vaccine of claim 41, wherein the lipid nanoparticle comprises a cationic lipid, a neutral and/or non-cationic lipid, a sterol, or any combination thereof.

43. The vaccine of claim 42, wherein the non-cationic lipid comprises a phospholipid.

44. The vaccine of claim 42 or 43, wherein the sterol comprises cholesterol or a modification or ester thereof.

45. The vaccine of any of claims 41-44, wherein the lipid nanoparticle comprises a polyethylene glycol (PEG)-lipid conjugate.

46. A method of treating or preventing a disease or disorder comprising administrating the composition of any of claims 1-20, the polynucleotide of any of claims 21-39, or the vaccine of any of claims 40-45 to a subject in need thereof.

47. The method of claim 46, wherein the disease or disorder comprises an infection.

48. The method of claim 47, wherein the infection comprises a coronavirus infection.

49. The method of claim 48, wherein the coronavirus is severe acute respiratory syndrome coronavirus-2 (SARS-CoV-2).

50. The method of any of claims 46-49, wherein the administering comprises an initial immunization and at least one subsequent immunization.

51. A method of inducing an immune response in a subject comprising administrating the composition of any of claims 1-20, the polynucleotide of any of claims 21-39, or the vaccine of any of claims 40-45.

52. The method of any of claims 46-51, wherein the subject is human.

53. Use of the composition of any of claims 1-20 or the polynucleotide of any of claims 21-39 in the manufacture of a medicament for the treatment or prevention a disease or disorder.

54. The use of claim 53, wherein the disease or disorder comprises an infection.

55. The use of claim 54, wherein the infection comprises a coronavirus infection.

56. The use of claim 55, wherein the coronavirus is severe acute respiratory syndrome coronavirus-2 (SARS-CoV-2).
